(19) United States
(12) Patent Application Publication
(10) Pub. No.: US 2020/0405873 A1
(43) Pub. Date: Dec. 31, 2020

(54) PYRROLOBENZODIAZEPINE-ANTIBODY CONJUGATES

(71) Applicants: MEDIMMUNE LIMITED, Cambridge (GB); ADC THERAPEUTICS S.A., Epalinges (CH)

(72) Inventors: Jay Marshall FEINGOLD, Murray Hill, NJ (US); Patricius Hendrikus Cornelis VAN BERKEL, Lausanne (CH)

(21) Appl. No.: 16/806,910

(22) Filed: Mar. 2, 2020

Related U.S. Application Data
(63) Continuation of application No. 15/746,913, filed on Jan. 23, 2018, now abandoned.

(30) Foreign Application Priority Data
Jul. 31, 2015 (GB) 1513607.0

(51) Int. Cl. A61K 47/68; A61P 35/02; A61K 31/5517; C07K 16/28; C07K 16/30

(52) U.S. Cl. CPC A61K 47/6803; A61K 47/6867; C07K 16/3061; A61K 31/5517; C07K 16/2896; A61P 35/02

(57) ABSTRACT

The present disclosure relates to the use of ADCs comprising anti-CD25 antibodies for in treating disorders characterized by the presence of CD25+ve cells.

### CROSS-REFERENCE TO RELATED APPLICATIONS

This application is a continuation of U.S. application Ser. No. 15/746,913, filed Jan. 23, 2018, now abandoned, which is a national phase application under 35 U.S.C. § 371 of PCT International Application No. PCT/EP2015/077689, filed Nov. 25, 2015, which claims priority to Great Britain Application No. 1513607.0, filed Jul. 31, 2015, the contents of each of which are incorporated by reference herein.

The present disclosure relates to particular uses of pyrrolobenzodiazepines (PBDs) having a labile C2 or N10 protecting group in the form of a linker to an antibody which binds to CD25.

### BACKGROUND

### Pyrrolobenzodiazepines

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994); Antonow, D. and Thurston, D. E., Chem. Rev. 2011 111 (4), 2815-2864). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102) (Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

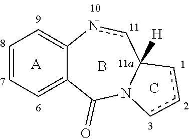

They differ in the number, type and position of substituents, in both their aromatic A rings and pyrrolo C rings, and in the degree of saturation of the C ring. In the B-ring there is either an imine (N═C), a carbinolamine(NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

A particularly advantageous pyrrolobenzodiazepine compound is described by Gregson et al. (Chem. Commun. 1999, 797-798) as compound 1, and by Gregson et al. (J. Med. Chem. 2001, 44, 1161-1174) as compound 4a. This compound, also known as SG2000, is shown below:

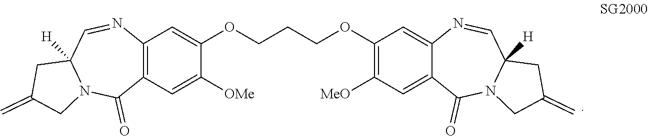

WO 2007/085930 describes the preparation of dimer PBD compounds having linker groups for connection to a cell binding agent, such as an antibody. The linker is present in the bridge linking the monomer PBD units of the dimer.

Dimer PBD compounds having linker groups for connection to a cell binding agent, such as an antibody, are described in WO 2011/130613, WO 2011/130616, WO2013/053873, WO2013/053871, WO2013/041606, WO2013/055993, WO2013/055990, WO2014/057073 and WO2015/052321. The linker in these compounds is attached to the PBD core via the C2 position, and are generally cleaved by action of an enzyme on the linker group. In WO 2011/130598, WO2013/055987, WO2014/057074 and WO2015/052322, the linker in these compounds is attached to one of the available N10 positions on the PBD core, and are generally cleaved by action of an enzyme on the linker group.

### Antibody-Drug Conjugates

Antibody therapy has been established for the targeted treatment of patients with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumor cells in the treatment of cancer, targets delivery of the drug moiety to tumors, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

Maximal efficacy with minimal toxicity is sought thereby. Efforts to design and refine ADC have focused on the selectivity of monoclonal antibodies (mAbs) as well as drug mechanism of action, drug-linking, drug/antibody ratio (loading), and drug-releasing properties (Junutula, et al., 2008b Nature Biotech., 26(8):925-932; Dornan et al (2009) Blood 114(13):2721-2729; U.S. Pat. Nos. 7,521,541; 7,723,485; WO2009/052249; McDonagh (2006) Protein Eng. Design & Sel. 19(7): 299-307; Doronina et al (2006) Bioconj. Chem. 17:114-124; Erickson et al (2006) Cancer Res. 66(8):1-8; Sanderson et al (2005) Clin. Cancer Res. 11:843-852; Jeffrey et al (2005) J. Med. Chem. 48:1344-1358; Hamblett et al (2004) Clin. Cancer Res. 10:7063-7070).

WO2014/57119 disclosed PBD dimers conjugated to an anti-CD25 antibody.

### In Vivo Anti-Tumour Activity

The in vivo anti-tumour activity of the conjugates described herein against CD25+ve human anaplastic large cell lymphoma (ALCL)-derived cell line Karpas299 xenograft model is demonstrated in WO 2014057119 A1; see, for example, FIG. 4 and the accompanying description.

### BRIEF DESCRIPTION OF THE DRAWINGS

FIG. 1 is a schematic representation and chemical structure of ADCT-301. Abbreviations: Ala, alanine; PABA, para-aminobenzoic acid; PEG, polyethylene glycol; TAC, T-cell activation antigen; Val, valine.

### DISCLOSURE

As described in more detail below, the present authors have found that ADCs as defined herein, when conjugated to anti-CD25 antibodies, are highly effective in treating disorders characterized by the presence of CD25+ve cells. Specifically, the present authors describe anti-CD25/PBD ADCs for use in treating CD25+ve Acute Myeloid Leukemias (AML), including relapsed or refractory CD25+ve Acute Myeloid Leukemias (rAML).

The provision of new treatments specifically targeted to cells expressing CD25 will provide additional therapeutic options for patients with AML and rAML. Accordingly, the present disclosure represents a significant contibution to the field of AML treatment.

Thus, a first aspect of the present disclosure provides a method of treating CD25+ve Acute Myeloid Leukemias (AML) in a subject,
said method comprising administering to the subject a conjugate of formula L-(D^{L})ₚ, where D^{L} is of formula I or II:

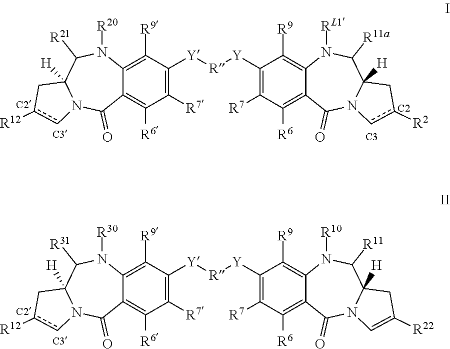

wherein:
L is an antibody (Ab) which binds to CD25;
p is an integer from 1 to 20;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

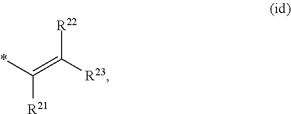

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

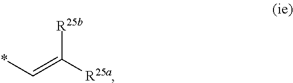

wherein one of R^{25a} and R^{25b} is H and the other is selected from: phenyl, which phenyl is optionally substituted by a group selected from halo, methyl, methoxy; pyridyl; and thiophenyl; and

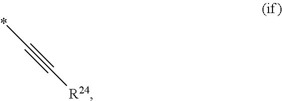

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

### R¹² is

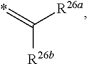

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;

### [Formula I]

R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C1-3 alkylene;
(ib) C₁₋₅ saturated aliphatic alkyl;
(ic) C₃₋₆ saturated cycloalkyl;

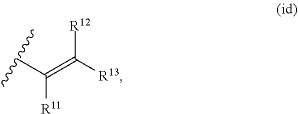

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

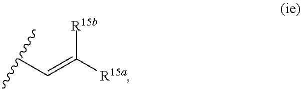

wherein one of R^{15a} and R^{15b} is H and the other is selected from: phenyl, which phenyl is optionally substituted by a group selected from halo, methyl, methoxy; pyridyl; and thiophenyl; and

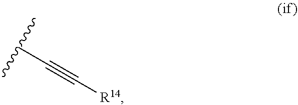

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

### R² is

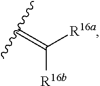

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula IIIa, formula IIIb or formula IIIc:

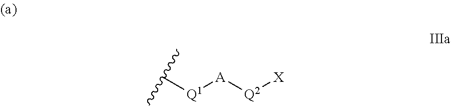

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

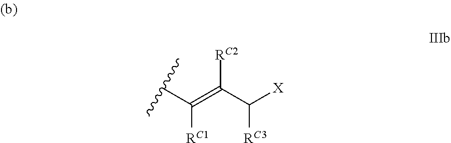

where;
R^{C1}, R^{C2} and R^{C3} are independently selected from H and unsubstituted C₁₋₂ alkyl;

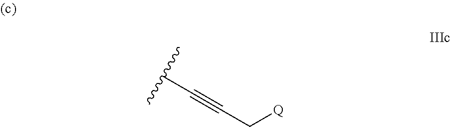

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

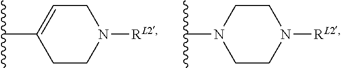

NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

Accordingly, the Conjugates comprise an antibody (Ab) which binds to CD25 covalently linked to at least one Drug unit by a Linker unit.

The drug loading is represented by p, the number of drug molecules per antibody. Drug loading may range from 1 to 20 Drug units (D^{L}) per antibody. For compositions, p represents the average drug loading of the Conjugates in the composition, and p ranges from 1 to 20.

In the practice of the disclosure, the drug moiety may be cleaved in vivo prior to or after internalisation by the target CD25+ve cells such as to release the PBD, wherein said PBD penetrates said CD25+ve cell causing cytoxicity thereto.

Preferably the cytotoxicity causes cell death.

In some embodiments the CD25+ve Acute Myeloid Leukemia (AML) is a relapsed or refractory CD25+ve Acute Myeloid Leukemia (rAML).

The term “relapsed or refractory AML (rAML)” as used herein refers to the diagnosis and classification of AML & rAML as per World Health Organization (WHO) classification of AML (Jaffe, 2001; Vardiman, 2002).

In some embodiments the ‘CD25+ve Acute Myeloid Leukemia (AML)’ may comprise both CD25+ve and CD25-ve cells. That is, by way of non-limiting example may a Leukemia in which the population of neoplastic CD-25+ve cells is heterogeneous.

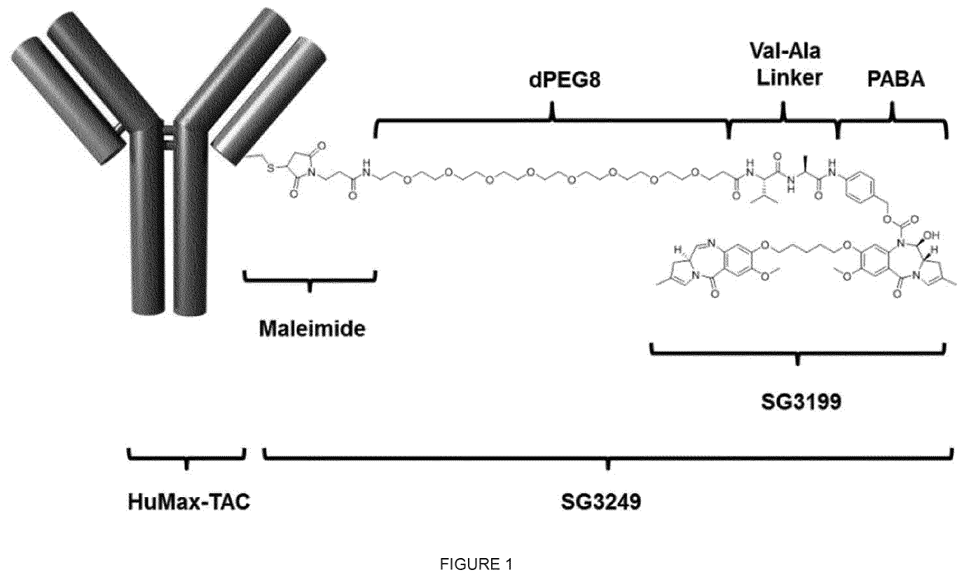

In some preferred embodiments the conjugate is “ADCT-301” as herein described (see structure in FIG. 1).

In a second aspect of the disclosure there is provided a method of causing cytotoxicity to (more preferably cell death of) a neoplastic CD25+ve cell which method comprises uses of a conjugate as defined in the first aspect of the disclosure. In some embodiments the cell is an AML blast cell. The method of this second aspect may be carried out in accordance with the first aspect.

In another aspect of the disclosure there is provided a method of selecting a subject for treatment with a conjugate as defined in the first aspect of the disclosure, which method comprises screening said subject to identify the presence of a neoplasm comprising CD25+ve cells. Patients are selected wherein such a neoplasm is present. Preferably the neoplasm is AML. In some embodiments the neoplasm is rAML.

In a third aspect of the disclosure there is provided a method of treating CD25+ve Acute Myeloid Leukemia (AML, such as AML or rAML) in a subject, said method comprising:
(i) identifying the presence in the subject of a neoplasm comprising CD25+ve cells;
(ii) administering to the subject a conjugate as defined in the first aspect of the disclosure.

Also provided are any of the conjugates described herein for use in any one of the methods of the disclosure, and use of such conjugates for the preparation of a medicament for use in any one of the methods of the disclosure.

Non-limiting examples of suitable diseases, neoplasms, and antibodies for the practice of the disclosure are described in more detail hereinafter.

### DETAILED DESCRIPTION

### AML

Acute myeloid leukaemia (AML) is a heterogeneous hematologic malignancy characterized by the clonal expansion of myeloid blasts in the peripheral blood, bone marrow, and/or other tissues. It is the most common leukaemia in adults and results in the largest number of deaths due to leukaemia in the U.S. The diagnosis will account for 38% of an estimated 54,270 new cases of leukaemia and 42% of estimated deaths due to leukaemia in 2015 (American Cancer Society, 2015). It is more common in older adults (median age at onset, 67 years), with 72% of cases diagnosed at ages 55 years and older.

The overall 5-year survival rate for AML is 26%, with younger patients (age <45 years) having a higher survival rate (>50%) compared with older patients (˜10% for patients 65-74 years) (National Cancer Institute, 2015). Cytotoxic induction therapy is associated with high rates of complete response/remission, especially for younger patients who are able to tolerate treatment. The discrepancy between the high response rates achieved with induction therapy and poor long-term survival is due to the inability to prevent or overcome disease relapse (Breems, 2006; Grimwade, 2001). The majority of patients with AML will eventually relapse or develop refractory disease. The prognosis for these patients is poor and most patients will die from progressive disease.

CD25, or TAC, is the 55 kilodalton (kDa) alpha chain of IL-2R (Burchill, 2007). In normal human tissue, expression of CD25 is mainly limited to activated T- and B-cells (Burchill, 2007; Buckner, 2010). It is not expressed on normal human hematopoietic stem cells (Seito, 2010), but has been demonstrated in subpopulations of chemotherapy-resistant human leukemic stem cells (LSC) (Ding, 2012). Survival of quiescent, chemotherapy-resistant LSCs may play a role in the development of relapsed or refractory disease in AML (Ding, 2012; Ishikawa, 2007).

Positive CD25 expression has been demonstrated in newly diagnosed and relapsed AML (Cerny, 2012; Gönen, 2012; Terwijn, 2009) and in late-stage myelodysplastic syndrome related AML (Miltiades, 2014). Expression of CD25 by AML blast cells is associated with adverse outcomes, including induction failure, relapse, and shortened overall survival (Cerny, 2012; Gönen, 2012; Miltiades, 2014; Terwijn, 2009). It is not yet understood what biological role, if any, CD25 may play in the development of adverse outcomes. However, the provision of treatment specifically targeted to cells expressing CD25 will provide additional therapeutic options for these patients.

“CD25+ve AML” as used herein is defined as determination of CD25 expression by ≥5% of blast cells obtained from bone marrow (aspirate or biopsy) or whole blood samples, as assessed via flow cytometry at an approved clinical laboratory

### Antibody Drug Conjugates

### Linker

The specified link between the PBD dimer and the antibody, in the present disclosure is preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Antibody that Binds to CD25

CD25 is also known as: IL2RA, RP11-536K7.1, IDDM10, IL2R, TCGFR, FIL-2 receptor subunit alpha; IL-2-RA; IL-2R subunit alpha; IL2-RA; TAC antigen; interleukin-2 receptor subunit alpha; p55

The CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589.

Antibodies that bind CD25 are described in:
U.S. Pat. No. 6,383,487 (Novartis/UCL: Baxilisimab [Simulect])
U.S. Pat. No. 6,521,230 (Novartis/UCL: Baxilisimab [Simulect])
   For example, an antibody having an antigen binding site comprises at least one domain which comprises CDR1 having the amino acid sequence in SEQ. ID. NO: 7, CDR2 having the amino acid sequence in SEQ. ID. NO: 8, and CDR3 having the amino acid sequence in SEQ. ID. NO: 9; or said CDR1, CDR2 and CDR3 taken in sequence as a whole comprise an amino acid sequence which is at least 90% identical to SEQ. ID. NOs: 7, 8 and 9 taken in sequence as a whole.
Daclizumab-Rech A J., et al Ann NY Acad Sci. 2009 September; 1174:99-106 (Roche)

Methods for distinguishing cells which are CD25+ve from those which are CD25-ve are well known in the art. Example techniques include by immunohistochemistry (Strauchen et al., al., Am. J Pathol. 126:506-512, 1987, FACS (Gaikwad et al., Int. J. Clin. Exp Pathol. 7: 6225-6230, 2014) or imaging of patients using SPECT/PET following administration of radiolabelled probes specific for CD25 (van Dort et al., Curr. Comput. Aided Drug Des. 4: 46-53, 2008). Such familiar methods may be used to identify patients with neoplasms suitable for targeting by the methods of the present disclosure.

In one aspect the antibody is an antibody that binds to CD25, the antibody comprising: a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5. In some embodiments the antibody comprises a VH domain having the sequence according to SEQ ID NO. 1.

The antibody may further comprise: a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8. In some embodiments the antibody further comprises a VL domain having the sequence according to SEQ ID NO. 2.

In some embodiments the antibody comprises a VH domain and a VL domain, the VH and VL domains having the sequences of SEQ ID NO. 1 paired with SEQ ID NO. 2.

The VH and VL domain(s) may pair so as to form an antibody antigen binding site that binds CD25.

In some embodiments the antibody is an intact antibody comprising a VH domain and a VL domain, the VH and VL domains having sequences of SEQ ID NO. 1 and SEQ ID NO. 2.

In some embodiments the antibody is a fully human monoclonal IgG1 antibody, preferably IgG1,κ.

In some embodiments the antibody is the AB12 antibody described in WO 2004/045512 (Genmab A/S), otherwise known as HuMax-TAC (see also VH and VL sequences disclosed herein as SEQ ID NOs. 1 & 2).

In an aspect the antibody is an antibody as described herein which has been modified (or further modified) as described below. In some embodiments the antibody is a humanised, deimmunised or resurfaced version of an antibody disclosed herein.

### Terminology

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, which is the ability to bind CD25 (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, orallotype (e.g. human G1 m1, G1m2, G1m3, non-G1 m1 [that, is any allotype other than G1 m1], G1m17, G2m23, G3m21, G3m28, G3m11, G3m5, G3m13, G3m14, G3m10, G3m15, G3m16, G3m6, G3m24, G3m26, G3m27, A2 ml, A2m2, Km1, Km2 and Km3) of immunoglobulin molecule. The immunoglobulins can be derived from any species, including human, murine, or rabbit origin.

As used herein, “binds CD25” is used to mean the antibody binds CD25 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 104, 105 or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567).

The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

### Modification of Antibodies

The antibodies disclosed herein may be modified. For example, to make them less immunogenic to a human subject. This may be achieved using any of a number of techniques familiar to the person skilled in the art. Some of these techniques are described in more detail below.

### Humanisation

Techniques to reduce the in vivo immunogenicity of a non-human antibody or antibody fragment include those termed “humanisation”.

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies. The expression “humanized antibody” also includes an immunoglobulin amino acid sequence variant or fragment thereof that comprises an FR having substantially the amino acid sequence of a human immunoglobulin and a CDR having substantially the amino acid sequence of a non-human immunoglobulin.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g. murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins.

There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

### CDR Grafting

In this technique, the humanized antibodies are human immunoglobulins (recipient antibody) in which residues from a complementary-determining region (CDR) of the recipient antibody are replaced by residues from a CDR of a non-human species (donor antibody) such as mouse, rat, camel, bovine, goat, or rabbit having the desired properties (in effect, the non-human CDRs are ‘grafted’ onto the human framework). In some instances, framework region (FR) residues of the human immunoglobulin are replaced by corresponding non-human residues (this may happen when, for example, a particular FR residue has significant effect on antigen binding).

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin.

### Guided Selection

The method consists of combining the V_{H} or V_{L} domain of a given non-human antibody specific for a particular epitope with a human V_{H} or V_{L} library and specific human V domains are selected against the antigen of interest. This selected human VH is then combined with a VL library to generate a completely human VHxVL combination. The method is described in Nature Biotechnology (N.Y.) 12, (1994) 899-903.

### Composite Antibodies

In this method, two or more segments of amino acid sequence from a human antibody are combined within the final antibody molecule. They are constructed by combining multiple human VH and VL sequence segments in combinations which limit or avoid human T cell epitopes in the final composite antibody V regions. Where required, T cell epitopes are limited or avoided by, exchanging V region segments contributing to or encoding a T cell epitope with alternative segments which avoid T cell epitopes. This method is described in US 2008/0206239 A1.

### Deimmunization

This method involves the removal of human (or other second species) T-cell epitopes from the V regions of the therapeutic antibody (or other molecule). The therapeutic antibodies V-region sequence is analysed for the presence of MHC class II-binding motifs by, for example, comparison with databases of MHC-binding motifs (such as the “motifs” database hosted at www.wehi.edu.au). Alternatively, MHC class II-binding motifs may be identified using computational threading methods such as those devised by Altuvia et al. (J. Mol. Biol. 249 244-250 (1995)); in these methods, consecutive overlapping peptides from the V-region sequences are testing for their binding energies to MHC class II proteins. This data can then be combined with information on other sequence features which relate to successfully presented peptides, such as amphipathicity, Rothbard motifs, and cleavage sites for cathepsin B and other processing enzymes.

Once potential second species (e.g. human) T-cell epitopes have been identified, they are eliminated by the alteration of one or more amino acids. The modified amino acids are usually within the T-cell epitope itself, but may also be adjacent to the epitope in terms of the primary or secondary structure of the protein (and therefore, may not be adjacent in the primary structure). Most typically, the alteration is by way of substitution but, in some circumstances amino acid addition or deletion will be more appropriate.

All alterations can be accomplished by recombinant DNA technology, so that the final molecule may be prepared by expression from a recombinant host using well established methods such as Site Directed Mutagenesis. However, the use of protein chemistry or any other means of molecular alteration is also possible.

### Resurfacing

This method involves:
(a) determining the conformational structure of the variable region of the non-human (e.g. rodent) antibody (or fragment thereof) by constructing a three-dimensional model of the non-human antibody variable region;
(b) generating sequence alignments using relative accessibility distributions from x-ray crystallographic structures of a sufficient number of non-human and human antibody variable region heavy and light chains to give a set of heavy and light chain framework positions wherein the alignment positions are identical in 98% of the sufficient number of non-human antibody heavy and light chains;
(c) defining for the non-human antibody to be humanized, a set of heavy and light chain surface exposed amino acid residues using the set of framework positions generated in step (b);
(d) identifying from human antibody amino acid sequences a set of heavy and light chain surface exposed amino acid residues that is most closely identical to the set of surface exposed amino acid residues defined in step (c), wherein the heavy and light chain from the human antibody are or are not naturally paired;
(e) substituting, in the amino acid sequence of the non-human antibody to be humanized, the set of heavy and light chain surface exposed amino acid residues defined in step (c) with the set of heavy and light chain surface exposed amino acid residues identified in step (d);
(f) constructing a three-dimensional model of the variable region of the non-human antibody resulting from the substituting specified in step (e);
(g) identifying, by comparing the three-dimensional models constructed in steps (a) and (f), any amino acid residues from the sets identified in steps (c) or (d), that are within 5 Angstroms of any atom of any residue of the complementarity determining regions of the non-human antibody to be humanized; and
(h) changing any residues identified in step (g) from the human to the original non-human amino acid residue to thereby define a non-human antibody humanizing set of surface exposed amino acid residues; with the proviso that step (a) need not be conducted first, but must be conducted prior to step (g).

### Superhumanization

The method compares the non-human sequence with the functional human germline gene repertoire. Those human genes encoding canonical structures identical or closely related to the non-human sequences are selected. Those selected human genes with highest homology within the CDRs are chosen as FR donors. Finally, the non-human CDRs are grafted onto these human FRs. This method is described in patent WO 2005/079479 A2.

### Human String Content Optimization

This method compares the non-human (e.g. mouse) sequence with the repertoire of human germline genes and the differences are scored as Human String Content (HSC) that quantifies a sequence at the level of potential MHC/T-cell epitopes. The target sequence is then humanized by maximizing its HSC rather than using a global identity measure to generate multiple diverse humanized variants (described in Molecular Immunology, 44, (2007) 1986-1998).

### Framework Shuffling

The CDRs of the non-human antibody are fused in-frame to cDNA pools encompassing all known heavy and light chain human germline gene frameworks. Humanised antibodies are then selected by e.g. panning of the phage displayed antibody library. This is described in Methods 36, 43-60 (2005).

### Preferred Embodiments

In some preferred embodiments the conjugate is “ADCT-301” as described below.

ADCT-301 is an antibody drug conjugate (ADC), composed of the human monoclonal antibody, HuMax®-TAC, directed against human cluster of differentiation 25 (CD25), and conjugated through a cleavable linker to SG3199, a pyrrolobenzodiazepine (PBD) dimer cytotoxin. PBD dimers are highly efficient anticancer drugs that bind in the minor groove of DNA and form highly cytotoxic DNA interstrand cross-links. The schematic representation of ADCT 301, and the different components that may be formed following the administration of this ADC to humans, are presented in FIG. 1.

The make-up of ADCT-301 includes:
HuMax®-TAC: A human monoclonal antibody of the IgG1, kappa isotype, specific for human CD25 and having VH and VL domains with the sequences specified in SEQ ID NOs. 1 and 2 disclosed herein.
SG3249: A PBD linker that comprises the PBD dimer SG3199 and all linker components, including the maleimide, 8-polyethylene glycol, a protease-sensitive valine-alanine linker and a para-aminobenzoic acid self immolative group (D^{L}=ConjE).
SG3199: A PBD dimer cytotoxin, which is a highly efficient anticancer drug due to its interstrand cross-linking, a consequence of its specifically designed strong binding to the minor groove of DNA.

The interleukin-2 receptor (IL-2R) is made up of three subunits: a (CD25), p (CD122) and γ (CD132). CD25, or T-cell activation antigen (TAC), is the alpha chain of IL-2R (Burchill, 2007, Buckner, 2010). ADCT-301 binds with picomolar affinity to human CD25. After binding and internalization, ADCT-301 is transported to the lysosomes, where the protease sensitive linker is cleaved and free PBD dimers are released inside the target cell. The released PBD dimers bind into the minor groove of DNA in a sequence-selective manner, and form highly cytotoxic DNA interstrand cross-links (Hartley, 2011[1]). The cross-links formed by PBD dimers are relatively non-distorting of the DNA structure, making them hidden to repair mechanisms, allowing for a longer effective period (Adair, 2012).

### Definitions

### Pharmaceutically Acceptable Cations

Examples of pharmaceutically acceptable monovalent and divalent cations are discussed in Berge, et al., J. Pharm. Sci., 66, 1-19 (1977), which is incorporated herein by reference.

The pharmaceutically acceptable cation may be inorganic or organic.

Examples of pharmaceutically acceptable monovalent inorganic cations include, but are not limited to, alkali metal ions such as Na⁺ and K⁺. Examples of pharmaceutically acceptable divalent inorganic cations include, but are not limited to, alkaline earth cations such as Ca²⁺ and Mg²⁺. Examples of pharmaceutically acceptable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

### Nomenclature

“+ve” is used herein to mean ‘positive’
“-ve” is used herein to mean ‘negative’

### Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:
   norcarane (C₇), norpinane (C₇), norbornane (C₇).
   C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. The term “C₅₋₇ aryl”, as used herein, pertains to a monovalent moiety obtained by removing a hydrogen atom from an aromatic ring atom of an aromatic compound, which moiety has from 5 to 7 ring atoms and the term “C₅₋₁₀ aryl”, as used herein, pertains to a monovalent moiety obtained by removing a hydrogen atom from an aromatic ring atom of an aromatic compound, which moiety has from 5 to 10 ring atoms. Preferably, each ring has from 5 to 7 ring atoms.

In this context, the prefixes (e.g. C₃₋₂₀, C₅₋₇, C₅₋₆, C₅₋₁₀, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

### Halo: —F, —Cl, —Br, and —I.

### Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.
Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).
Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).
Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).
Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).
Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).
Oxo (keto, -one): ═O.
Thione (thioketone): ═S.
Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, =NEt, and ═NPh.
Formyl (carbaldehyde, carboxaldehyde): —C(═O)H.
Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).
Carboxy (carboxylic acid): —C(═O)OH.
Thiocarboxy (thiocarboxylic acid): —C(═S)SH.
Thiolocarboxy (thiolocarboxylic acid): —C(═O)SH.
Thionocarboxy (thionocarboxylic acid): —C(═S)OH.
Imidic acid: —C(═NH)OH.
Hydroxamic acid: —C(═NOH)OH.
Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.
Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.
Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.
Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.
Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.
Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.
Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

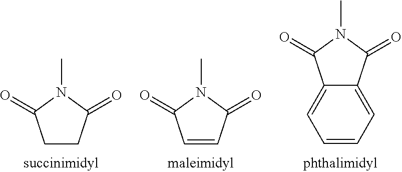

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.
Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

### Guanidino: —NH—C(═NH)NH₂.

Tetrazolyl: a five membered aromatic ring having four nitrogen atoms and one carbon atom,

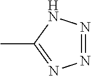

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and =NEt.
Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

### Nitro: —NO₂.

### Nitroso: —NO.

### Azido: —N₃.

Cyano (nitrile, carbonitrile): —CN.

### Isocyano: —NC.

### Cyanato: —OCN.

### Isocyanato: —NCO.

Thiocyano (thiocyanato): —SCN.
Isothiocyano (isothiocyanato): —NCS.
Sulfhydryl (thiol, mercapto): —SH.
Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.
Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.
Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.
Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).
Sulfinic acid (sulfino): —S(═O)OH, —SO₂H.
Sulfonic acid (sulfo): —S(═O)₂OH, —SO₃H.
Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).
Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).
Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.
Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).
Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.
Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.
Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.
Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.
Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.
Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

### Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.
Phosphonic acid (phosphono): —P(═O)(OH)₂.
Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.
Phosphoric acid (phosphonooxy): —OP(═O)(OH)₂.
Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.
Phosphorous acid: —OP(OH)₂.
Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.
Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.
Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

### Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below. Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

Carbamate nitrogen protecting group: the term “carbamate nitrogen protecting group” pertains to a moiety which masks the nitrogen in the imine bond, and these are well known in the art. These groups have the following structure:

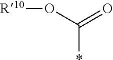

wherein R′¹⁰ is R as defined above. A large number of suitable groups are described on pages 503 to 549 of Greene, T. W. and Wuts, G. M., Protective Groups in Organic Synthesis, 3^{rd} Edition, John Wiley & Sons, Inc., 1999, which is incorporated herein by reference.

Hemi-aminal nitrogen protecting group: the term “hemi-aminal nitrogen protecting group” pertains to a group having the following structure:

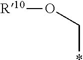

wherein R′¹⁰ is R as defined above. A large number of suitable groups are described on pages 633 to 647 as amide protecting groups of Greene, T. W. and Wuts, G. M., Protective Groups in Organic Synthesis, 3^{rd} Edition, John Wiley & Sons, Inc., 1999, which is incorporated herein by reference.

The groups Carbamate nitrogen protecting group and Hemi-aminal nitrogen protecting group may be jointly termed a “nitrogen protecting group for synthesis”.

### Conjugates

The present disclosure relates to a conjugate comprising a PBD compound connected to the antibody via a Linker Unit.

In one embodiment, the conjugate comprises the antibody connected to a spacer connecting group, the spacer connected to a trigger, the trigger connected to a self-immolative linker, and the self-immolative linker connected to the N10 position of the PBD compound. Such a conjugate is illustrated below:

where Ab is the antibody as defined above and PBD is a pyrrolobenzodiazepine compound (D), as described herein. The illustration shows the portions that correspond to R^{L′}, A, L¹ and L² in certain embodiments of the disclosure. R^{L′} may be either R^{L1′} or R^{L2′}. D is D^{L} with R^{L1′} or R^{L2′} removed.

In the preferred embodiments, the conjugate allows the release of an active PBD compound that does not retain any part of the linker. There is no stub present that could affect the reactivity of the PBD compound.

The linker attaches the antibody to the PBD drug moiety D through covalent bond(s). The linker is a bifunctional or multifunctional moiety which can be used to link one or more drug moiety (D) and an antibody unit (Ab) to form antibody-drug conjugates (ADC). The linker (R^{L′}) may be stable outside a cell, i.e. extracellular, or it may be cleavable by enzymatic activity, hydrolysis, or other metabolic conditions. Antibody-drug conjugates (ADC) can be conveniently prepared using a linker having reactive functionality for binding to the drug moiety and to the antibody. A cysteine thiol, or an amine, e.g. N-terminus or amino acid side chain such as lysine, of the antibody (Ab) can form a bond with a functional group of a linker or spacer reagent, PBD drug moiety (D) or drug-linker reagent (D^{L}, D-R^{L}), where R^{L} can be R^{L1} or R^{L2}.

The linkers of the ADC preferably prevent aggregation of ADC molecules and keep the ADC freely soluble in aqueous media and in a monomeric state.

The linkers of the ADC are preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targetted site; and (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p 234-242).

In another embodiment, the linker may be substituted with groups which modulate aggregation, solubility or reactivity. For example, a sulfonate substituent may increase water solubility of the reagent and facilitate the coupling reaction of the linker reagent with the antibody or the drug moiety, or facilitate the coupling reaction of Ab-L with D^{L}, or D^{L}-L with Ab, depending on the synthetic route employed to prepare the ADC.

In one embodiment, L-R^{L′} is a group:

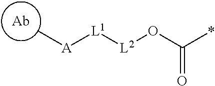

where the asterisk indicates the point of attachment to the Drug Unit (D), Ab is the antibody (L), L¹ is a linker, A is a connecting group connecting L¹ to the antibody, L² is a covalent bond or together with —OC(═O)— forms a self-immolative linker, and L¹ or L² is a cleavable linker.

L¹ is preferably the cleavable linker, and may be referred to as a trigger for activation of the linker for cleavage.

The nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidising conditions may also find use in the present disclosure.

L¹ may comprise a contiguous sequence of amino acids. The amino acid sequence may be the target substrate for enzymatic cleavage, thereby allowing release of L-R^{L′} from the N10 position.

In one embodiment, L¹ is cleavable by the action of an enzyme. In one embodiment, the enzyme is an esterase or a peptidase.

In one embodiment, L² is present and together with —C(═O)O— forms a self-immolative linker. In one embodiment, L² is a substrate for enzymatic activity, thereby allowing release of L-R^{L′} from the N10 position.

In one embodiment, where L¹ is cleavable by the action of an enzyme and L² is present, the enzyme cleaves the bond between L¹ and L².

L¹ and L², where present, may be connected by a bond selected from:
—C(═O)NH—,
—C(═O)O—,
—NHC(═O)—,
—OC(═O)—,
—OC(═O)O—,
—NHC(═O)O—,
—OC(═O)NH—, and
—NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

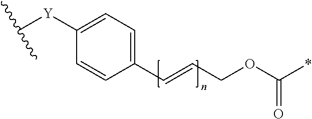

where the asterisk indicates the point of attachment to the N10 position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents as described herein. In one embodiment, the phenylene group is optionally substituted with halo, NO₂, R or OR.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

The self-immolative linker will allow for release of the protected compound when a remote site is activated, proceeding along the lines shown below (for n=0):

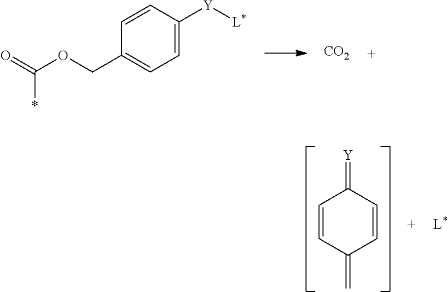

where L* is the activated form of the remaining portion of the linker. These groups have the advantage of separating the site of activation from the compound being protected. As described above, the phenylene group may be optionally substituted.

In one embodiment described herein, the group L* is a linker L¹ as described herein, which may include a dipeptide group.

In another embodiment, —C(═O)O— and L² together form a group selected from:

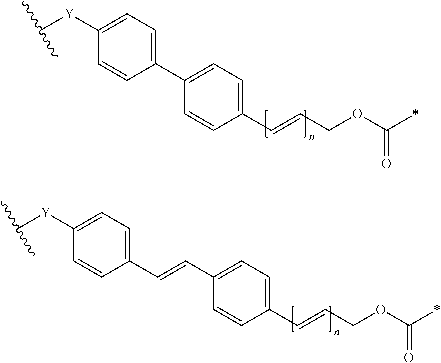

where the asterisk, the wavy line, Y, and n are as defined above. Each phenylene ring is optionally substituted with one, two or three substituents as described herein. In one embodiment, the phenylene ring having the Y substituent is optionally substituted and the phenylene ring not having the Y substituent is unsubstituted. In one embodiment, the phenylene ring having the Y substituent is unsubstituted and the phenylene ring not having the Y substituent is optionally substituted.

In another embodiment, —C(═O)O— and L² together form a group selected from:

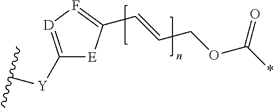

where the asterisk, the wavy line, Y, and n are as defined above, E is O, S or NR, D is N, CH, or CR, and F is N, CH, or CR.

In one embodiment, D is N.

In one embodiment, D is CH.

In one embodiment, E is O or S.

In one embodiment, F is CH.

In a preferred embodiment, the linker is a cathepsin labile linker.

In one embodiment, L¹ comprises a dipeptide The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-,
-Trp-Cit-
   where Cit is citrulline.

Preferably, the group —X₁—X₂— in dipeptide, —NH—X₁—X₂—CO—, is selected from:
-Phe-Lys-,
-Val-Ala-,
-Val-Lys-,
-Ala-Lys-,
-Val-Cit-.

Most preferably, the group —X₁—X₂— in dipeptide, —NH—X₁—X₂—CO—, is -Phe-Lys- or -Val-Ala-.

Other dipeptide combinations may be used, including those described by Dubowchik et al., Bioconjugate Chemistry, 2002, 13,855-869, which is incorporated herein by reference.

In one embodiment, the amino acid side chain is derivatised, where appropriate. For example, an amino group or carboxy group of an amino acid side chain may be derivatised.

In one embodiment, an amino group NH₂ of a side chain amino acid, such as lysine, is a derivatised form selected from the group consisting of NHR and NRR′.

In one embodiment, a carboxy group COOH of a side chain amino acid, such as aspartic acid, is a derivatised form selected from the group consisting of COOR, CONH₂, CONHR and CONRR′.

In one embodiment, the amino acid side chain is chemically protected, where appropriate. The side chain protecting group may be a group as discussed below in relation to the group R^{L}. The present authors have established that protected amino acid sequences are cleavable by enzymes. For example, it has been established that a dipeptide sequence comprising a Boc side chain-protected Lys residue is cleavable by cathepsin.

Protecting groups for the side chains of amino acids are well known in the art and are described in the Novabiochem Catalog. Additional protecting group strategies are set out in Protective Groups in Organic Synthesis, Greene and Wuts.

Possible side chain protecting groups are shown below for those amino acids having reactive side chain functionality:
Arg: Z, Mtr, Tos;
Asn: Trt, Xan;
Asp: Bzl, t-Bu;
Cys: Acm, Bzl, Bzl-OMe, Bzl-Me, Trt;
Glu: Bzl, t-Bu;
Gln: Trt, Xan;
His: Boc, Dnp, Tos, Trt;
Lys: Boc, Z—Cl, Fmoc, Z, Alloc;
Ser: Bzl, TBDMS, TBDPS;
Thr: Bz;
Trp: Boc;
Tyr: Bzl, Z, Z—Br.

In one embodiment, the side chain protection is selected to be orthogonal to a group provided as, or as part of, a capping group, where present. Thus, the removal of the side chain protecting group does not remove the capping group, or any protecting group functionality that is part of the capping group.

In other embodiments of the disclosure, the amino acids selected are those having no reactive side chain functionality. For example, the amino acids may be selected from: Ala, Gly, Ile, Leu, Met, Phe, Pro, and Val.

In one embodiment, the dipeptide is used in combination with a self-immolative linker. The self-immolative linker may be connected to —X₂—.

Where a self-immolative linker is present, —X₂— is connected directly to the self-immolative linker. Preferably the group —X₂—CO— is connected to Y, where Y is NH, thereby forming the group —X₂—CO—NH—.

—NH—X₁— is connected directly to A. A may comprise the functionality —CO— thereby to form an amide link with —X₁—.

In one embodiment, L¹ and L² together with —OC(═O)— comprise the group NH—X₁—X₂—CO-PABC-. The PABC group is connected directly to the N10 position. Preferably, the self-immolative linker and the dipeptide together form the group —NH-Phe-Lys-CO—NH-PABC-, which is illustrated below:

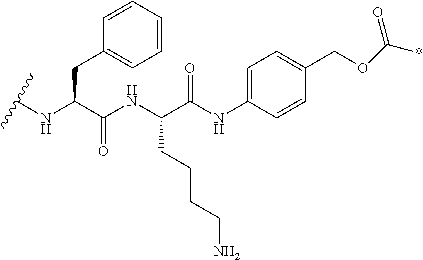 [SMILES: *N[C@@H](Cc1ccccc1)C(=O)N[C@@H](CCCCN)C(=O)Nc1ccc(COC(*)=O)cc1]

where the asterisk indicates the point of attachment to the N10 position, and the wavy line indicates the point of attachment to the remaining portion of the linker L¹ or the point of attachment to A. Preferably, the wavy line indicates the point of attachment to A. The side chain of the Lys amino acid may be protected, for example, with Boc, Fmoc, or Alloc, as described above.

Alternatively, the self-immolative linker and the dipeptide together form the group —NH-Val-Ala-CO—NH-PABC-, which is illustrated below:

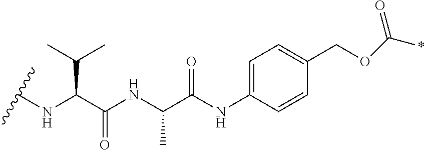 [SMILES: *N[C@H](C(=O)N[C@@H](C)C(=O)Nc1ccc(COC(*)=O)cc1)C(C)C]

where the asterisk and the wavy line are as defined above.

Alternatively, the self-immolative linker and the dipeptide together form the group —NH-Val-Cit-CO—NH-PABC-, which is illustrated below:

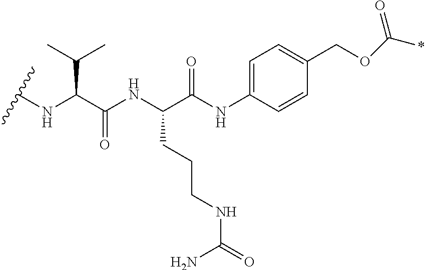 [SMILES: *N[C@H](C(=O)N[C@@H](CCCNC(N)=O)C(=O)Nc1ccc(COC(*)=O)cc1)C(C)C]

where the asterisk and the wavy line are as defined above.

In one embodiment, A is a covalent bond. Thus, L¹ and the antibody are directly connected. For example, where L¹ comprises a contiguous amino acid sequence, the N-terminus of the sequence may connect directly to the antibody.

Thus, where A is a covalent bond, the connection between the antibody and L¹ may be selected from:
—C(═O)NH—,
—C(═O)O—,
—NHC(═O)—,
—OC(═O)—,
—OC(═O)O—,
—NHC(═O)O—,
—OC(═O)NH—,
—NHC(═O)NH—,
—C(═O)NHC(═O)—,
—S—,
—S—S—,
—CH₂C(═O)—, and
═N—NH—.

An amino group of L¹ that connects to the antibody may be the N-terminus of an amino acid or may be derived from an amino group of an amino acid side chain, for example a lysine amino acid side chain.

An carboxyl group of L¹ that connects to the antibody may be the C-terminus of an amino acid or may be derived from a carboxyl group of an amino acid side chain, for example a glutamic acid amino acid side chain.

A hydroxyl group of L¹ that connects to the antibody may be derived from a hydroxyl group of an amino acid side chain, for example a serine amino acid side chain.

A thiol group of L¹ that connects to the antibody may be derived from a thiol group of an amino acid side chain, for example a serine amino acid side chain.

The comments above in relation to the amino, carboxyl, hydroxyl and thiol groups of L¹ also apply to the antibody.

In one embodiment, L² together with —OC(═O)— represents:

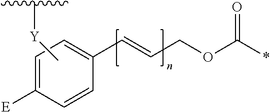

where the asterisk indicates the point of attachment to the N10 position, the wavy line indicates the point of attachment to L¹, n is 0 to 3, Y is a covalent bond or a functional group, and E is an activatable group, for example by enzymatic action or light, thereby to generate a self-immolative unit. The phenylene ring is optionally further substituted with one, two or three substituents as described herein. In one embodiment, the phenylene group is optionally further substituted with halo, NO₂, R or OR. Preferably n is 0 or 1, most preferably 0.

E is selected such that the group is susceptible to activation, e.g. by light or by the action of an enzyme. E may be —NO₂ or glucoronic acid. The former may be susceptible to the action of a nitroreductase, the latter to the action of a β-glucoronidase.

In this embodiment, the self-immolative linker will allow for release of the protected compound when E is activated, proceeding along the lines shown below (for n=0):

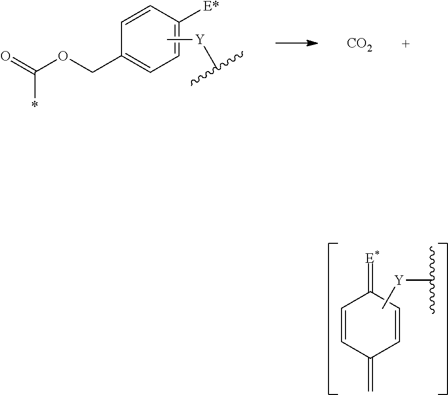

where the asterisk indicates the point of attachment to the N10 position, E* is the activated form of E, and Y is as described above. These groups have the advantage of separating the site of activation from the compound being protected. As described above, the phenylene group may be optionally further substituted.

The group Y may be a covalent bond to L¹.

The group Y may be a functional group selected from:
—C(═O)—
—NH—
—O—
—C(═O)NH—,
—C(═O)O—,
—NHC(═O)—,
—OC(═O)—,
—OC(═O)O—,
—NHC(═O)O—,
—OC(═O)NH—,
—NHC(═O)NH—,
—NHC(═O)NH,
—C(═O)NHC(═O)—, and
—S—.

Where L¹ is a dipeptide, it is preferred that Y is —NH— or —C(═O)—, thereby to form an amide bond between L¹ and Y. In this embodiment, the dipeptide sequence need not be a substrate for an enzymatic activity.

In another embodiment, A is a spacer group. Thus, L¹ and the antibody are indirectly connected.

L¹ and A may be connected by a bond selected from:
—C(═O)NH—,
—C(═O)O—,
—NHC(═O)—,
—OC(═O)—,
—OC(═O)O—,
—NHC(═O)O—,
—OC(═O)NH—, and
—NHC(═O)NH—.

In one embodiment, the group A is:

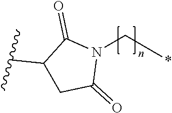

where the asterisk indicates the point of attachment to L¹, the wavy line indicates the point of attachment to the antibody, and n is 0 to 6. In one embodiment, n is 5.

In one embodiment, the group A is:

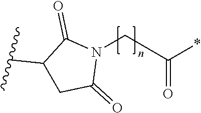

where the asterisk indicates the point of attachment to L¹, the wavy line indicates the point of attachment to the antibody, and n is 0 to 6. In one embodiment, n is 5.

In one embodiment, the group A is:

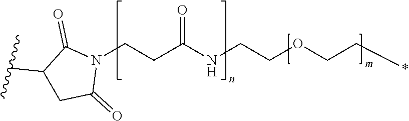

where the asterisk indicates the point of attachment to L¹, the wavy line indicates the point of attachment to the antibody, n is 0 or 1, and m is 0 to 30. In a preferred embodiment, n is 1 and m is 0 to 10, 1 to 8, preferably 4 to 8, and most preferably 4 or 8. In another embodiment, m is 10 to 30, and preferably 20 to 30. Alternatively, m is 0 to 50. In this embodiment, m is preferably 10-40 and n is 1.

In one embodiment, the group A is:

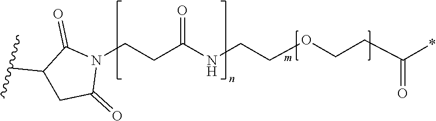

where the asterisk indicates the point of attachment to L¹, the wavy line indicates the point of attachment to the antibody, n is 0 or 1, and m is 0 to 30. In a preferred embodiment, n is 1 and m is 0 to 10, 1 to 8, preferably 4 to 8, and most preferably 4 or 8. In another embodiment, m is 10 to 30, and preferably 20 to 30. Alternatively, m is 0 to 50. In this embodiment, m is preferably 10-40 and n is 1.

In one embodiment, the connection between the antibody and A is through a thiol residue of the antibody and a maleimide group of A.

In one embodiment, the connection between the antibody and A is:

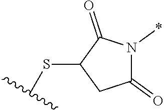 [SMILES: *SC1CC(=O)N(*)C1=O]

where the asterisk indicates the point of attachment to the remaining portion of A and the wavy line indicates the point of attachment to the remaining portion of the antibody. In this embodiment, the S atom is typically derived from the antibody.

In each of the embodiments above, an alternative functionality may be used in place of the maleimide-derived group shown below:

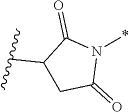 [SMILES: *C1CC(=O)N(*)C1=O]

where the wavy line indicates the point of attachment to the antibody as before, and the asterisk indicates the bond to the remaining portion of the A group.

In one embodiment, the maleimide-derived group is replaced with the group:

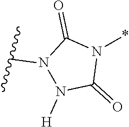 [SMILES: *n1[nH]c(=O)n(*)c1=O]

where the wavy line indicates point of attachment to the antibody, and the asterisk indicates the bond to the remaining portion of the A group.

In one embodiment, the maleimide-derived group is replaced with a group, which optionally together with the antibody, is selected from:
—C(═O)NH—,
—C(═O)O—,
—NHC(═O)—,
—OC(═O)—,
—OC(═O)O—,
—NHC(═O)O—,
—OC(═O)NH—,
—NHC(═O)NH—,
—NHC(═O)NH,
—C(═O)NHC(═O)—,
—S—,
—S—S—,
—CH₂C(═O)—
—C(═O)CH₂—,
═N—NH—, and
—NH—N═.

In one embodiment, the maleimide-derived group is replaced with a group, which optionally together with the antibody, is selected from:

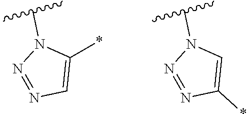 [SMILES: *c1cnnn1* ; *c1cn(*)nn1]

where the wavy line indicates either the point of attachment to the antibody or the bond to the remaining portion of the A group, and the asterisk indicates the other of the point of attachment to the antibody or the bond to the remaining portion of the A group.

Other groups suitable for connecting L¹ to the antibody are described in WO 2005/082023.

In one embodiment, the Connecting Group A is present, the Trigger L¹ is present and Self-Immolative Linker L² is absent. Thus, L¹ and the Drug unit are directly connected via a bond. Equivalently in this embodiment, L² is a bond. This may be particularly relevant when D^{L} is of Formula II.

L¹ and D may be connected by a bond selected from:
—C(═O)N<,
—C(═O)O—,
—NHC(═O)—,
—OC(═O)—,
—OC(═O)O—,
—NHC(═O)O—,
—OC(═O)N<, and
—NHC(═O)N<,
   where N< or O— are part of D.

In one embodiment, L¹ and D are preferably connected by a bond selected from:
—C(═O)N<, and
—NHC(═O)—.

In one embodiment, L¹ comprises a dipeptide and one end of the dipeptide is linked to D. As described above, the amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, the group —X₁—X₂— in dipeptide, —NH—X₁—X₂—CO—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-, and
-Trp-Cit-;
   where Cit is citrulline. In such a dipeptide, —NH— is the amino group of X₁, and CO is the carbonyl group of X₂.

Preferably, the group-X₁—X₂— in dipeptide, —NH—X₁—X₂—CO—, is selected from:
-Phe-Lys-,
-Val-Ala-,
-Val-Lys-,
-Ala-Lys-, and
-Val-Cit-.

Most preferably, the group —X₁—X₂— in dipeptide, —NH—X₁—X₂—CO—, is -Phe-Lys- or -Val-Ala-.

Other dipeptide combinations of interest include:
-Gly-Gly-,
-Pro-Pro-, and
-Val-Glu-.

Other dipeptide combinations may be used, including those described above.

In one embodiment, L¹-D is:

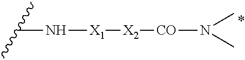

where —NH—X₁—X₂—CO is the dipeptide, —N< is part of the Drug unit, the asterisk indicates the points of attachment to the remainder of the Drug unit, and the wavy line indicates the point of attachment to the remaining portion of L¹ or the point of attachment to A. Preferably, the wavy line indicates the point of attachment to A.

In one embodiment, the dipeptide is valine-alanine and L¹-D is:

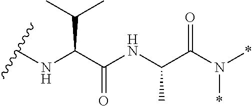 [SMILES: *N[C@H](C(=O)N[C@@H](C)C(=O)N(*)*)C(C)C]

where the asterisks, —N< and the wavy line are as defined above.

In one embodiment, the dipeptide is phenylalnine-lysine and L¹-D is:

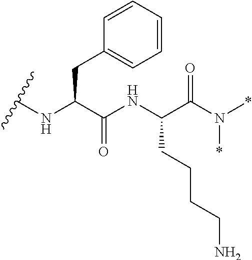 [SMILES: *N[C@@H](Cc1ccccc1)C(=O)N[C@@H](CCCCN)C(=O)N(*)*]

where the asterisks, —N< and the wavy line are as defined above.

In one embodiment, the dipeptide is valine-citrulline.

In one embodiment, the groups A-L¹ are:

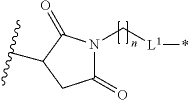

where the asterisk indicates the point of attachment to L² or D, the wavy line indicates the point of attachment to the Ligand unit, and n is 0 to 6. In one embodiment, n is 5.

In one embodiment, the groups A-L¹ are:

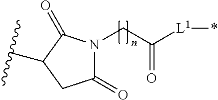

where the asterisk indicates the point of attachment to L² or D, the wavy line indicates the point of attachment to the Ligand unit, and n is 0 to 6. In one embodiment, n is 5.

In one embodiment, the groups A-L¹ are:

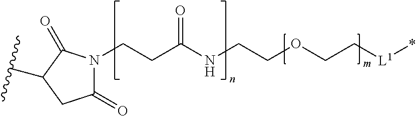

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A-L¹ are:

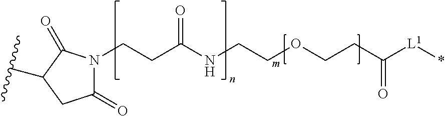

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 7, preferably 3 to 7, most preferably 3 or 7.

In one embodiment, the groups A-L¹ are:

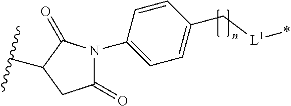

where the asterisk indicates the point of attachment to L² or D, the wavy line indicates the point of attachment to the Ligand unit, and n is 0 to 6. In one embodiment, n is 5.

In one embodiment, the groups A-L¹ are:

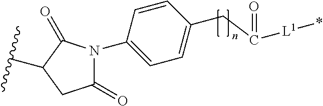

where the asterisk indicates the point of attachment to L² or D, the wavy line indicates the point of attachment to the Ligand unit, and n is 0 to 6. In one embodiment, n is 5.

In one embodiment, the groups A-L¹ are:

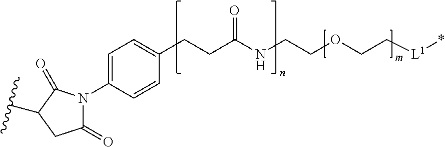

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A-L¹ is:

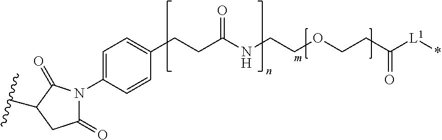

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A-L¹ are:

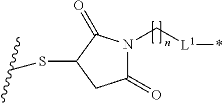

where the asterisk indicates the point of attachment to L² or D, S is a sulfur group of the Ligand unit, the wavy line indicates the point of attachment to the rest of the Ligand unit, and n is 0 to 6. In one embodiment, n is 5.

In one embodiment, the group A-L¹ are:

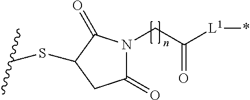

where the asterisk indicates the point of attachment to L² or D, S is a sulfur group of the Ligand unit, the wavy line indicates the point of attachment to the remainder of the Ligand unit, and n is 0 to 6. In one embodiment, n is 5.

In one embodiment, the groups A¹-L¹ are:

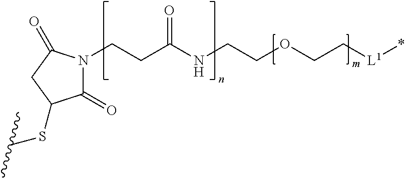

where the asterisk indicates the point of attachment to L² or D, S is a sulfur group of the Ligand unit, the wavy line indicates the point of attachment to the remainder of the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A¹-L¹ are:

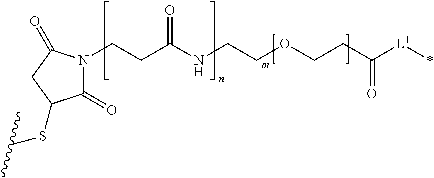

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 7, preferably 4 to 8, most preferably 4 or 8.

In one embodiment, the groups A¹-L¹ are:

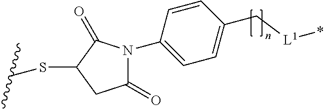

where the asterisk indicates the point of attachment to L² or D, the wavy line indicates the point of attachment to the remainder of the Ligand unit, and n is 0 to 6. In one embodiment, n is 5.

In one embodiment, the groups A¹-L¹ are:

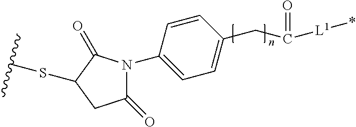

where the asterisk indicates the point of attachment to L² or D, the wavy line indicates the point of attachment to the remainder of the Ligand unit, and n is 0 to 6. In one embodiment, n is 5.

In one embodiment, the groups A¹-L¹ are:

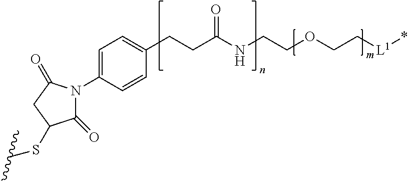

where the asterisk indicates the point of attachment to L² or D, the wavy line indicates the point of attachment to the remainder of the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A¹-L¹ are:

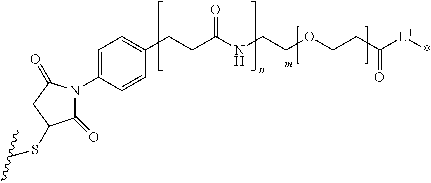

where the asterisk indicates the point of attachment to L² or D, the wavy line indicates the point of attachment to the remainder of the Ligand unit, n is 0 or 1, and m is 0 to 30. In a preferred embodiment, n is 1 and m is 0 to 10, 1 to 8, preferably 4 to 8, most preferably 4 or 8.

The group R^{L′} is derivable from the group R^{L}. The group R^{L} may be converted to a group R^{L′} by connection of an antibody to a functional group of R^{L}. Other steps may be taken to convert R^{L} to R^{L′}. These steps may include the removal of protecting groups, where present, or the installation of an appropriate functional group.

### R^{L}

Linkers can include protease-cleavable peptidic moieties comprising one or more amino acid units. Peptide linker reagents may be prepared by solid phase or liquid phase synthesis methods (E. Schröder and K. Lübke, The Peptides, volume 1, pp 76-136 (1965) Academic Press) that are well known in the field of peptide chemistry, including t-BOC chemistry (Geiser et al “Automation of solid-phase peptide synthesis” in Macromolecular Sequencing and Synthesis, Alan R. Liss, Inc., 1988, pp. 199-218) and Fmoc/HBTU chemistry (Fields, G. and Noble, R. (1990) “Solid phase peptide synthesis utilizing 9-fluoroenylmethoxycarbonyl amino acids”, Int. J. Peptide Protein Res. 35:161-214), on an automated synthesizer such as the Rainin Symphony Peptide Synthesizer (Protein Technologies, Inc., Tucson, Ariz.), or Model 433 (Applied Biosystems, Foster City, Calif.).

Exemplary amino acid linkers include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzymes, for example, a tumor-associated protease, cathepsin B, C and D, or a plasmin protease.

Amino acid side chains include those occurring naturally, as well as minor amino acids and non-naturally occurring amino acid analogs, such as citrulline. Amino acid side chains include hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl, as well as the following structures:

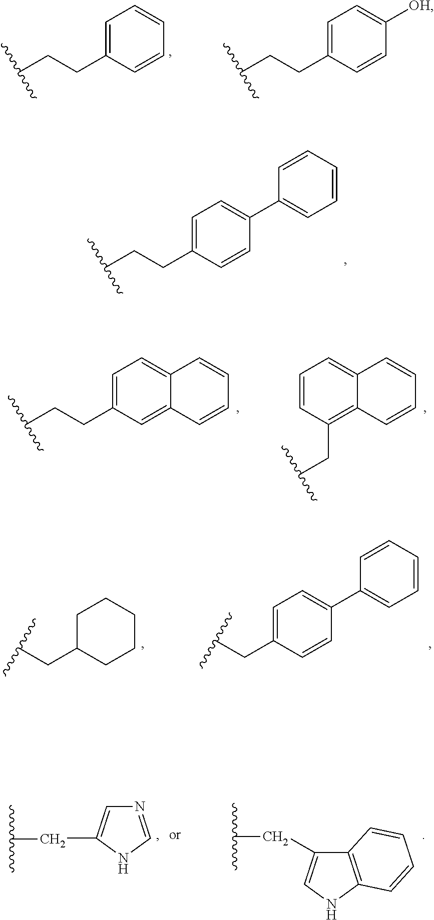

When the amino acid side chains include other than hydrogen (glycine), the carbon atom to which the amino acid side chain is attached is chiral. Each carbon atom to which the amino acid side chain is attached is independently in the (S) or (R) configuration, or a racemic mixture. Drug-linker reagents may thus be enantiomerically pure, racemic, or diastereomeric.

In exemplary embodiments, amino acid side chains are selected from those of natural and non-natural amino acids, including alanine, 2-amino-2-cyclohexylacetic acid, 2-amino-2-phenylacetic acid, arginine, asparagine, aspartic acid, cysteine, glutamine, glutamic acid, glycine, histidine, isoleucine, leucine, lysine, methionine, norleucine, phenylalanine, proline, serine, threonine, tryptophan, tyrosine, valine, γ-aminobutyric acid, α,α-dimethyl γ-aminobutyric acid, β,β-dimethyl γ-aminobutyric acid, ornithine, and citrulline (Cit).

An exemplary valine-citrulline (val-cit or vc) dipeptide linker reagent useful for constructing a linker-PBD drug moiety intermediate for conjugation to an antibody, having a para-aminobenzylcarbamoyl (PAB) self-immolative spacer has the structure:

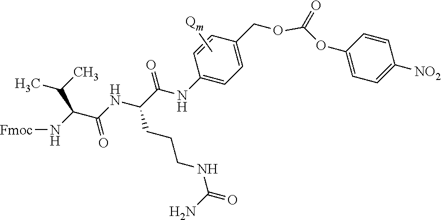

where Q is C₁-C₈ alkyl, —O—(C₁-C₈ alkyl), -halogen, —NO₂ or —CN; and m is an integer ranging from 0-4.

An exemplary phe-lys(Mtr) dipeptide linker reagent having a p-aminobenzyl group can be prepared according to Dubowchik, et al. (1997) Tetrahedron Letters, 38:5257-60, and has the structure:

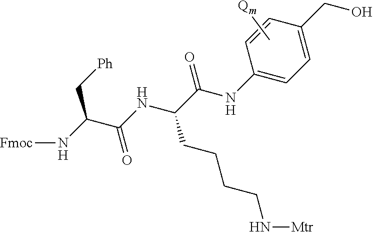

where Mtr is mono-4-methoxytrityl, Q is C₁-C₈ alkyl, —O—(C₁-C₈ alkyl), -halogen, —NO₂ or —CN; and m is an integer ranging from 0-4.

The “self-immolative linker” PAB (para-aminobenzyloxycarbonyl), attaches the drug moiety to the antibody in the antibody drug conjugate (Carl et al (1981) J. Med. Chem. 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other examples of self-immolative spacers besides PAB include, but are not limited to: (i) aromatic compounds that are electronically similar to the PAB group such as 2-aminoimidazol-5-methanol derivatives (Hay et al. (1999) Bioorg. Med. Chem. Lett. 9:2237), thiazoles (U.S. Pat. No. 7,375,078), multiple, elongated PAB units (de Groot et al (2001) J. Org. Chem. 66:8815-8830); and ortho or para-aminobenzylacetals; and (ii) homologated styryl PAB analogs (U.S. Pat. No. 7,223,837). Spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Elimination of amine-containing drugs that are substituted at glycine (Kingsbury et al (1984) J. Med. Chem. 27:1447) are also examples of self-immolative spacers useful in ADC.

In one embodiment, a valine-citrulline dipeptide PAB analog reagent has a 2,6 dimethyl phenyl group and has the structure:

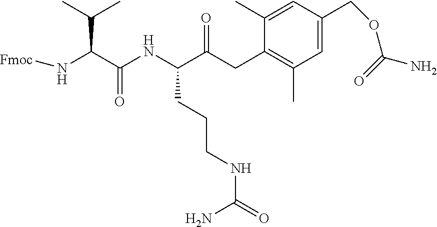

Linker reagents useful for the antibody drug conjugates of the disclosure include, but are not limited to: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and bis-maleimide reagents: DTME, BMB, BMDB, BMH, BMOE, 1,8-bis-maleimidodiethyleneglycol (BM(PEO)₂), and 1,11-bis-maleimidotriethyleneglycol (BM(PEO)₃), which are commercially available from Pierce Biotechnology, Inc., ThermoScientific, Rockford, Ill., and other reagent suppliers. Bis-maleimide reagents allow the attachment of a free thiol group of a cysteine residue of an antibody to a thiol-containing drug moiety, label, or linker intermediate, in a sequential or concurrent fashion. Other functional groups besides maleimide, which are reactive with a thiol group of an antibody, PBD drug moiety, or linker intermediate include iodoacetamide, bromoacetamide, vinyl pyridine, disulfide, pyridyl disulfide, isocyanate, and isothiocyanate.

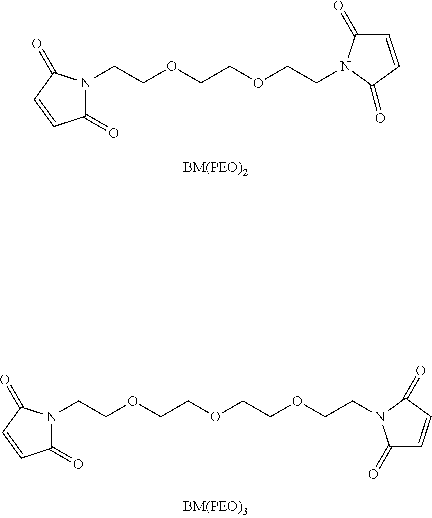 [SMILES: O=C1C=CC(=O)N1CCOCCOCCN1C(=O)C=CC1=O ; O=C1C=CC(=O)N1CCOCCOCCOCCN1C(=O)C=CC1=O]

Other embodiments of linker reagents are: N-succinimidyl-4-(2-pyridylthio)pentanoate (SPP), N-succinimidyl-3-(2-pyridyldithio) propionate (SPDP, Carlsson et al (1978) Biochem. J. 173:723-737), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Useful linker reagents can also be obtained via other commercial sources, such as Molecular Biosciences Inc. (Boulder, Colo.), or synthesized in accordance with procedures described in Toki et al (2002) J. Org. Chem. 67:1866-1872; U.S. Pat. No. 6,214,345; WO 02/088172; US 2003130189; US2003096743; WO 03/026577; WO 03/043583; and WO 04/032828.

The Linker may be a dendritic type linker for covalent attachment of more than one drug moiety through a branching, multifunctional linker moiety to an antibody (US 2006/116422; US 2005/271615; de Groot et al (2003) Angew. Chem. Int. Ed. 42:4490-4494; Amir et al (2003) Angew. Chem. Int. Ed. 42:4494-4499; Shamis et al (2004) J. Am. Chem. Soc. 126:1726-1731; Sun et al (2002) Bioorganic & Medicinal Chemistry Letters 12:2213-2215; Sun et al (2003) Bioorganic & Medicinal Chemistry 11:1761-1768; King et al (2002) Tetrahedron Letters 43:1987-1990). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic or branched linker.

One exemplary embodiment of a dendritic type linker has the structure:

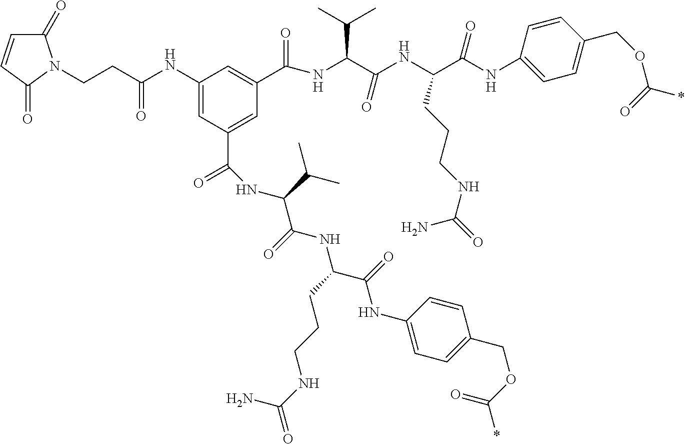 [SMILES: *C(=O)OCc1ccc(NC(=O)[C@H](CCCNC(N)=O)NC(=O)[C@@H](NC(=O)c2cc(NC(=O)CCN3C(=O)C=CC3=O)cc(C(=O)N[C@H](C(=O)N[C@@H](CCCNC(N)=O)C(=O)Nc3ccc(COC(*)=O)cc3)C(C)C)c2)C(C)C)cc1]

where the asterisk indicate the point of attachment to the N10 position of a PBD moiety.

### R^{c}, Capping Group

The conjugate of the first aspect of the disclosure may have a capping group R^{C} at the N10 position (R²⁰).

The group R^{C} is removable from the N10 position of the PBD moiety to leave an N10-C11 imine bond, a carbinolamine, a substituted carbinolamine, where QR¹¹ is OSO₃M, a bisulfite adduct, a thiocarbinolamine, a substituted thiocarbinolamine, or a substituted carbinalamine.

In one embodiment, R^{C}, may be a protecting group that is removable to leave an N10-C11 imine bond, a carbinolamine, a substituted cabinolamine, or, where QR¹¹ is OSO₃M, a bisulfite adduct. In one embodiment, R^{C} is a protecting group that is removable to leave an N10-C11 imine bond.

The group R^{c} is intended to be removable under the same conditions as those required for the removal of the group R¹⁰, for example to yield an N10-C11 imine bond, a carbinolamine and so on. The capping group acts as a protecting group for the intended functionality at the N10 position. The capping group is intended not to be reactive towards an antibody. For example, R^{C} is not the same as R^{L1′}

In one embodiment, the group R^{C} is removable under the conditions that cleave the linker R^{L1′}. Thus, in one embodiment, the capping group is cleavable by the action of an enzyme. R^{C} may be an N10 protecting group, such as those groups described in the authors' earlier application, WO 00/12507. In one embodiment, R^{C} is a therapeutically removable nitrogen protecting group, as defined in the authors' earlier application, WO 00/12507.

In one embodiment, R^{C} is a carbamate protecting group.

In one embodiment, the carbamate protecting group is selected from:
Alloc, Fmoc, Boc, Troc, Teoc, Psec, Cbz and PNZ.

Optionally, the carbamate protecting group is further selected from Moc.

In one embodiment, R^{C} is a linker group R^{L1′} lacking the functional group for connection to the antibody.

This application is particularly concerned with those R^{C} groups which are carbamates.

In one embodiment, R^{C} is a group:

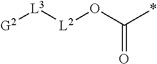

where the asterisk indicates the point of attachment to the N10 position, G² is a terminating group, L³ is a covalent bond or a cleavable linker L¹, L² is a covalent bond or together with OC(═O) forms a self-immolative linker.

Where L³ and L² are both covalent bonds, G² and OC(═O) together form a carbamate protecting group as defined above.

L² is as defined above in relation to R^{L1′}.

Various terminating groups are described below, including those based on well known protecting groups.

In one embodiment L³ is a cleavable linker L¹, and L², together with OC(═O), forms a self-immolative linker. In this embodiment, G² is Ac (acetyl) or Moc, or a carbamate protecting group selected from:
Alloc, Fmoc, Boc, Troc, Teoc, Psec, Cbz and PNZ.

Optionally, the carbamate protecting group is further selected from Moc.

In another embodiment, G² is an acyl group —C(═O)G³, where G³ is selected from alkyl (including cycloalkyl, alkenyl and alkynyl), heteroalkyl, heterocyclyl and aryl (including heteroaryl and carboaryl). These groups may be optionally substituted. The acyl group together with an amino group of L³ or L², where appropriate, may form an amide bond. The acyl group together with a hydroxy group of L³ or L², where appropriate, may form an ester bond.

In one embodiment, G³ is heteroalkyl. The heteroalkyl group may comprise polyethylene glycol. The heteroalkyl group may have a heteroatom, such as O or N, adjacent to the acyl group, thereby forming a carbamate or carbonate group, where appropriate, with a heteroatom present in the group L³ or L², where appropriate.

In one embodiment, G³ is selected from NH₂, NHR and NRR′. Preferably, G³ is NRR′.

In one embodiment G² is the group:

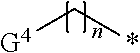

where the asterisk indicates the point of attachment to L³, n is 0 to 6 and G⁴ is selected from OH, OR, SH, SR, COOR, CONH₂, CONHR, CONRR′, NH₂, NHR, NRR′, NO₂, and halo. The groups OH, SH, NH₂ and NHR are protected. In one embodiment, n is 1 to 6, and preferably n is 5. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Most preferably G⁴ is OMe.

In one embodiment, the group G² is:

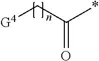

where the asterisk indicates the point of attachment to L³, and n and G⁴ are as defined above.

In one embodiment, the group G² is:

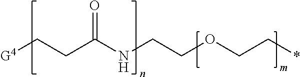

where the asterisk indicates the point of attachment to L³, n is 0 or 1, m is 0 to 50, and G⁴ is selected from OH, OR, SH, SR, COOR, CONH₂, CONHR, CONRR′, NH₂, NHR, NRR′, NO₂, and halo. In a preferred embodiment, n is 1 and m is 0 to 10, 1 to 2, preferably 4 to 8, and most preferably 4 or 8. In another embodiment, n is 1 and m is 10 to 50, preferably 20 to 40. The groups OH, SH, NH₂ and NHR are protected. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Preferably G⁴ is OMe.

In one embodiment, the group G² is:

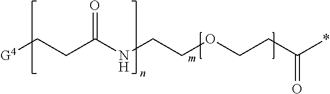

where the asterisk indicates the point of attachment to L³, and n, m and G⁴ are as defined above.

In one embodiment, the group G² is:

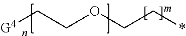

where n is 1-20, m is 0-6, and G⁴ is selected from OH, OR, SH, SR, COOR, CONH₂, CONHR, CONRR′, NH₂, NHR, NRR′, NO₂, and halo. In one embodiment, n is 1-10. In another embodiment, n is 10 to 50, preferably 20 to 40. In one embodiment, n is 1. In one embodiment, m is 1. The groups OH, SH, NH₂ and NHR are protected. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Preferably G⁴ is OMe.

In one embodiment, the group G² is:

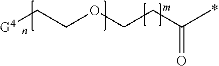

where the asterisk indicates the point of attachment to L³, and n, m and G⁴ are as defined above.

In each of the embodiments above G⁴ may be OH, SH, NH₂ and NHR. These groups are preferably protected.

In one embodiment, OH is protected with Bzl, TBDMS, or TBDPS.

In one embodiment, SH is protected with Acm, Bzl, Bzl-OMe, Bzl-Me, or Trt.

In one embodiment, NH₂ or NHR are protected with Boc, Moc, Z—Cl, Fmoc, Z, or Alloc.

In one embodiment, the group G² is present in combination with a group L³, which group is a dipeptide.

The capping group is not intended for connection to the antibody. Thus, the other monomer present in the dimer serves as the point of connection to the antibody via a linker. Accordingly, it is preferred that the functionality present in the capping group is not available for reaction with an antibody. Thus, reactive functional groups such as OH, SH, NH₂, COOH are preferably avoided. However, such functionality may be present in the capping group if protected, as described above.

### EMBODIMENTS

In some embodiments, D^{L} is selected from the group comprising:

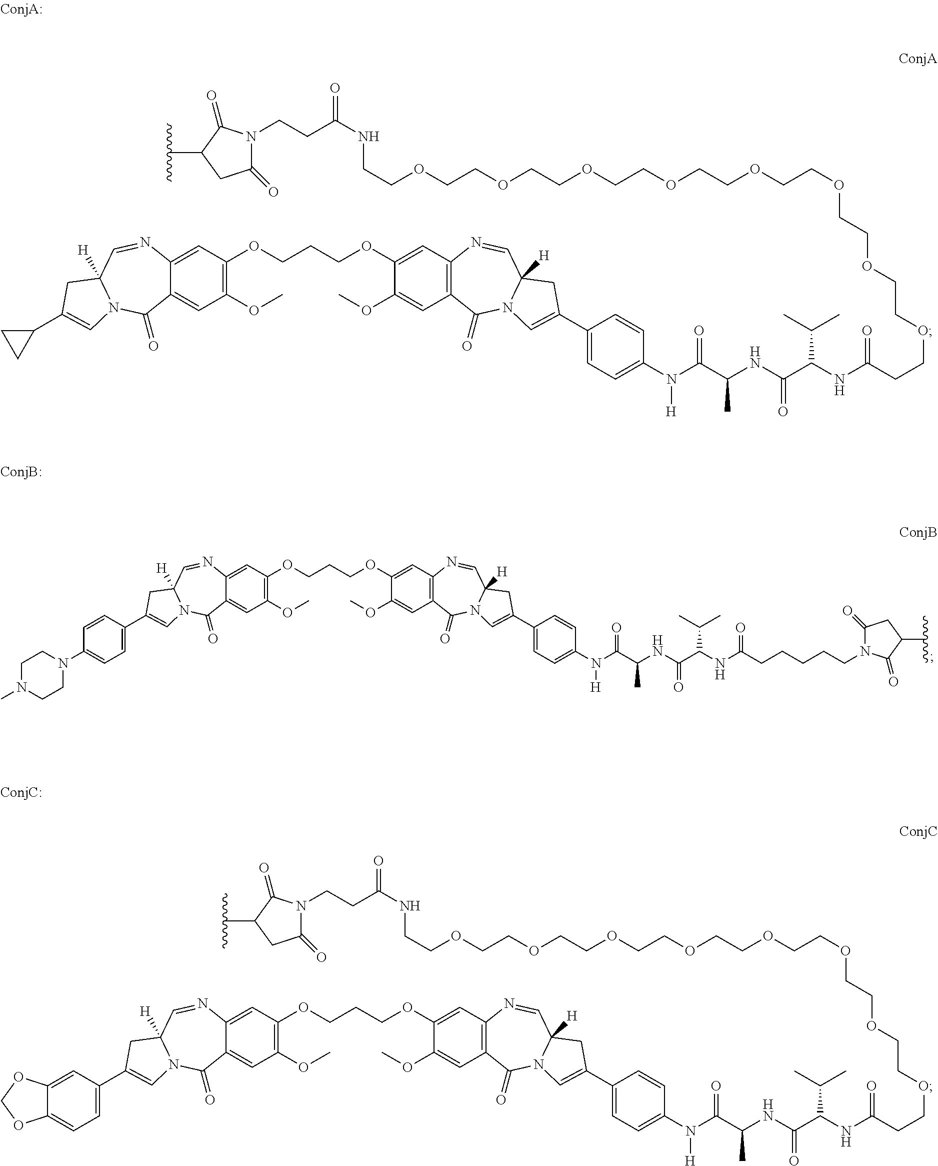

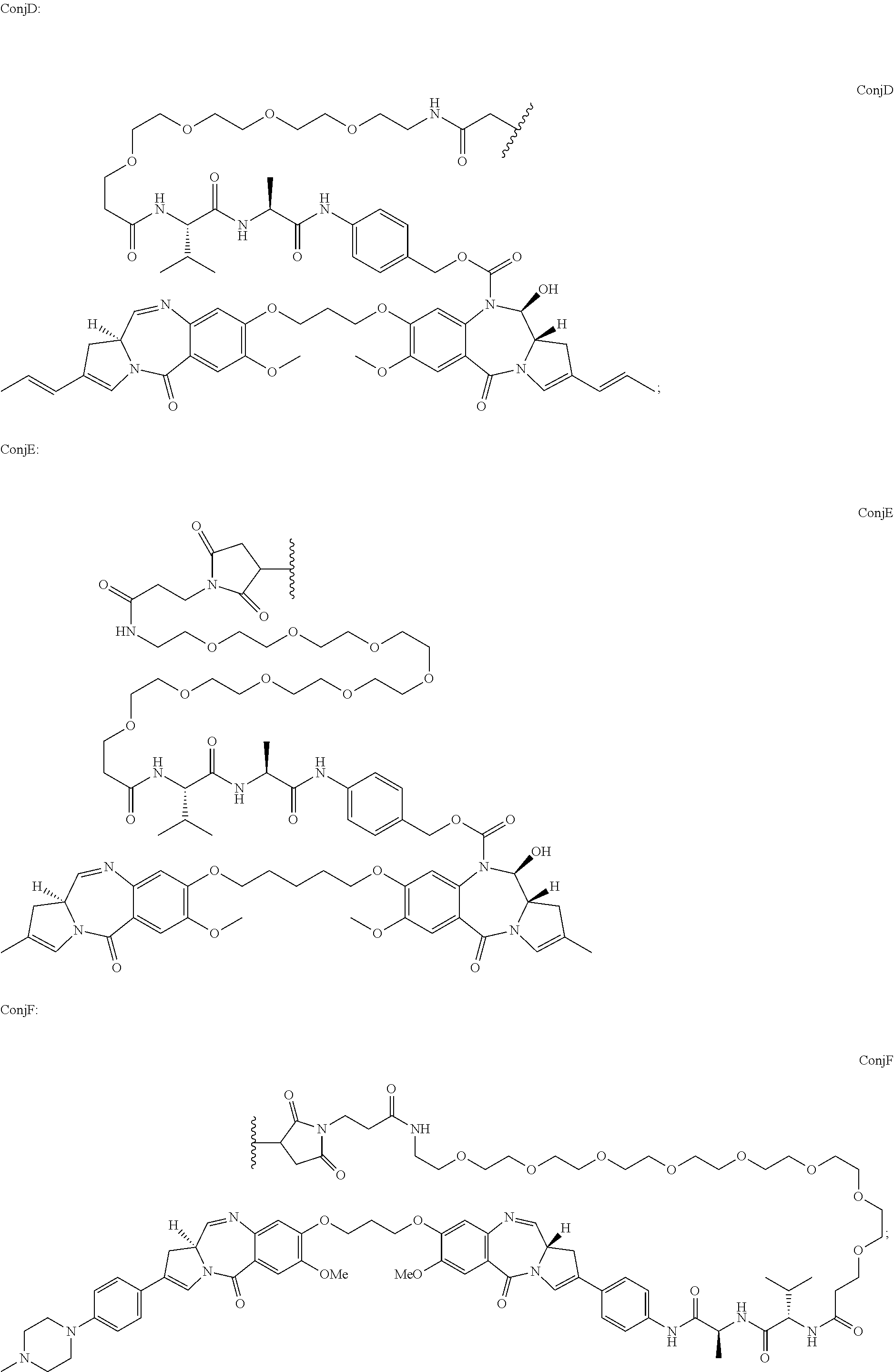

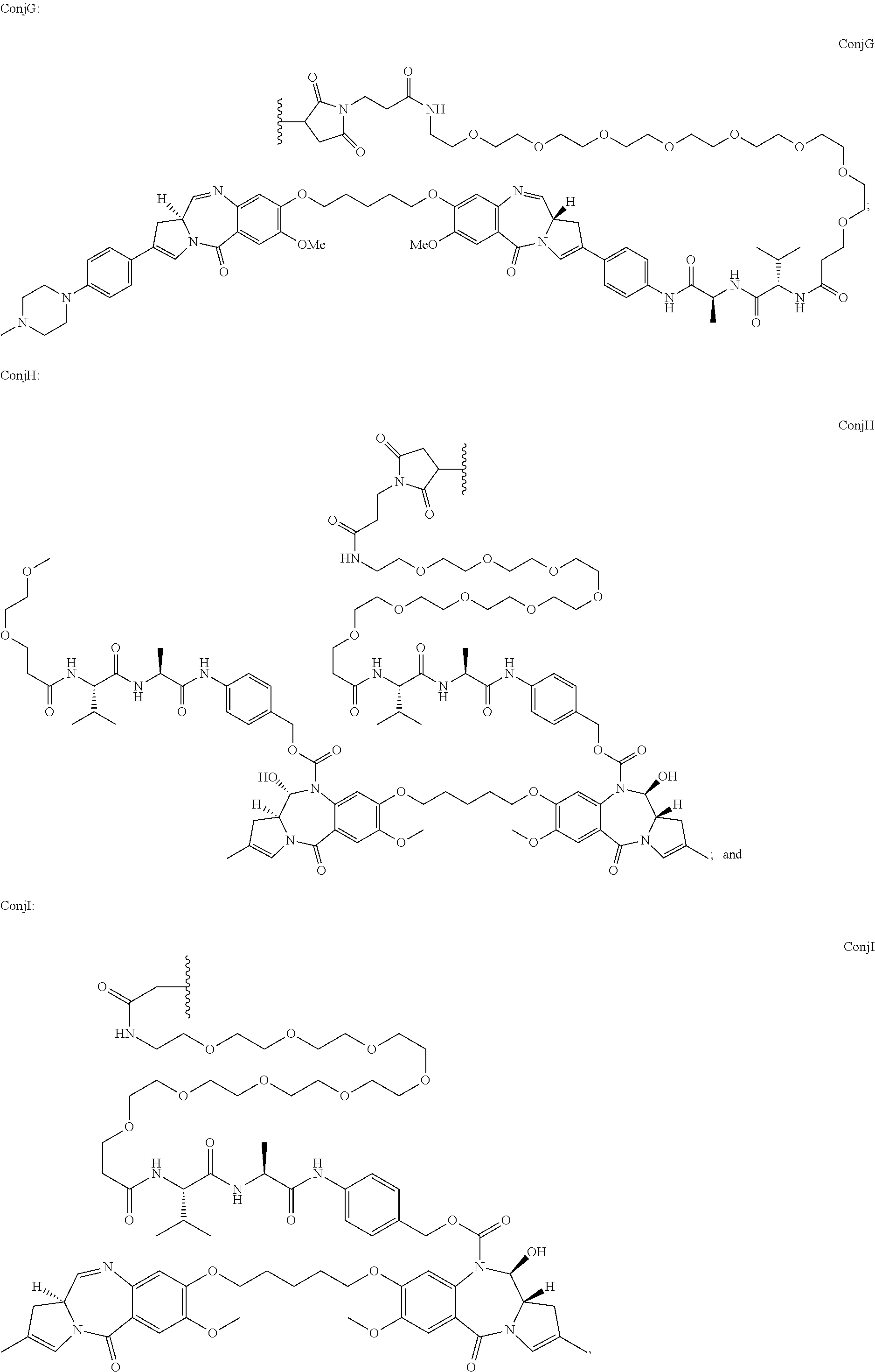

### Drug Loading

The drug loading is the average number of PBD drugs per antibody, e.g. antibody. Where the compounds of the disclosure are bound to cysteines, drug loading may range from 1 to 8 drugs (D^{L}) per antibody, i.e. where 1, 2, 3, 4, 5, 6, 7, and 8 drug moieties are covalently attached to the antibody. Compositions of conjgates include collections of antibodies, conjugated with a range of drugs, from 1 to 8. Where the compounds of the disclosure are bound to lysines, drug loading may range from 1 to 20 drugs (D^{L}) per antibody, although an upper limit of 10 or 8 may be preferred. Compositions of conjgates include collections of antibodies, conjugated with a range of drugs, from 1 to 20, 1 to 10 or 1 to 8.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p >5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the drug-linker intermediate (D-L) or linker reagent. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate (D-L) or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with linker reagents or the drug-linker reagents of the present disclosure which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Alternatively, site-specific conjugation can be achieved by engineering antibodies to contain unnatural amino acids in their heavy and/or light chains as described by Axup et al. ((2012), Proc Natl Acad Sci USA. 109(40):16101-16116). The unnatural amino acids provide the additional advantage that orthogonal chemistry can be designed to attach the linker reagent and drug.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per antibody is in the range 1 to 20. In some embodiments the range is selected from 1 to 8, 2 to 8, 2 to 6, 2 to 4, and 4 to 8.

In some embodiments, there is one dimer pyrrolobenzodiazepine group per antibody.

### Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

### Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄+) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

### Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

The disclosure includes compounds where a solvent adds across the imine bond of the PBD moiety, which is illustrated below where the solvent is water or an alcohol (R^{A}OH, where R^{A} is C₁₋₄ alkyl):

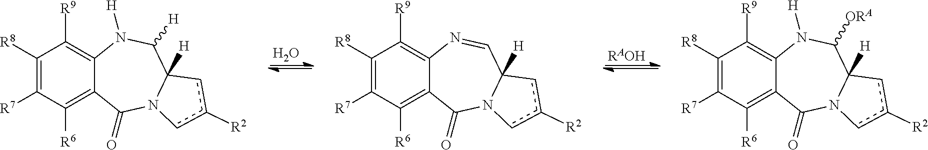

These forms can be called the carbinolamine and carbinolamine ether forms of the PBD (as described in the section relating to R¹⁰ above). The balance of these equilibria depend on the conditions in which the compounds are found, as well as the nature of the moiety itself.

These particular compounds may be isolated in solid form, for example, by lyophilisation.

### Isomers

Certain compounds of the disclosure may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and I or (+) and (−) are employed to designate the sign of rotation of plane-polarized light by the compound, with (−) or I meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

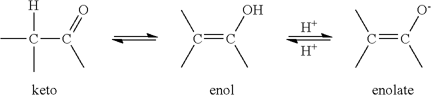

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

### Biological Activity

### In Vitro Cell Proliferation Assays

Generally, the cytotoxic or cytostatic activity of an antibody-drug conjugate (ADC) may be measured or confirmed by: exposing mammalian cells (including both those having and lacking receptor proteins) to the antibody of the ADC in a cell culture medium; culturing the cells for a period from about 6 hours to about 5 days; and measuring cell viability. Cell-based in vitro assays are used to measure viability (proliferation), cytotoxicity, and induction of apoptosis (caspase activation) of an ADC of the disclosure.

The in vitro potency of antibody-drug conjugates can be measured by a cell proliferation assay. The CellTiter-Glo® Luminescent Cell Viability Assay is a commercially available (Promega Corp., Madison, Wis.), homogeneous assay method based on the recombinant expression of Coleoptera luciferase (U.S. Pat. Nos. 5,583,024; 5,674,713 and 5,700,670). This cell proliferation assay determines the number of viable cells in culture based on quantitation of the ATP present, an indicator of metabolically active cells (Crouch et al (1993) J. Immunol. Meth. 160:81-88; U.S. Pat. No. 6,602,677). The CellTiter-Glo® Assay is conducted in 96 well format, making it amenable to automated high-throughput screening (HTS) (Cree et al (1995) AntiCancer Drugs 6:398-404). The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells/well in a 384-well format in 10 minutes after adding reagent and mixing. The cells may be treated continuously with ADC, or they may be treated and separated from ADC. Generally, cells treated briefly, i.e. 3 hours, showed the same potency effects as continuously treated cells.

The homogeneous “add-mix-measure” format results in cell lysis and generation of a luminescent signal proportional to the amount of ATP present. The amount of ATP is directly proportional to the number of cells present in culture. The CellTiter-Glo® Assay generates a “glow-type” luminescent signal, produced by the luciferase reaction, which has a half-life generally greater than five hours, depending on cell type and medium used. Viable cells are reflected in relative luminescence units (RLU). The substrate, Beetle Luciferin, is oxidatively decarboxylated by recombinant firefly luciferase with concomitant conversion of ATP to AMP and generation of photons.

The in vitro potency of antibody-drug conjugates can also be measured by a cytotoxicity assay. Cultured adherent cells are washed with PBS, detached with trypsin, diluted in complete medium, containing 10% FCS, centrifuged, re-suspended in fresh medium and counted with a haemocytometer. Suspension cultures are counted directly. Monodisperse cell suspensions suitable for counting may require agitation of the suspension by repeated aspiration to break up cell clumps.

The cell suspension is diluted to the desired seeding density and dispensed (100 μl per well) into black 96 well plates. Plates of adherent cell lines are incubated overnight to allow adherence. Suspension cell cultures can be used on the day of seeding.

A stock solution (1 ml) of ADC (20 μg/ml) is made in the appropriate cell culture medium. Serial 10-fold dilutions of stock ADC are made in 15 ml centrifuge tubes by serially transferring 100 μl to 900 μl of cell culture medium.

Four replicate wells of each ADC dilution (100 μl) are dispensed in 96-well black plates, previously plated with cell suspension (100 μl), resulting in a final volume of 200 μl. Control wells receive cell culture medium (100 μl).

If the doubling time of the cell line is greater than 30 hours, ADC incubation is for 5 days, otherwise a four day incubation is done.

At the end of the incubation period, cell viability is assessed with the Alamar blue assay. AlamarBlue (Invitrogen) is dispensed over the whole plate (20 μl per well) and incubated for 4 hours. Alamar blue fluorescence is measured at excitation 570 nm, emission 585 nm on the Varioskan flash plate reader. Percentage cell survival is calculated from the mean fluorescence in the ADC treated wells compared to the mean fluorescence in the control wells.

The in vitro cytotoxicity for CD25+ve SUDHL1 cells of the the conjugates described herein is demonstrated in WO 2014057119 A1; see, for example, FIG. 3 and the accompanying description.

### In Vivo Anti-Tumour Activity

The in vivo anti-tumour activity of the the conjugates described herein against CD25+ve human anaplastic large cell lymphoma (ALCL)-derived cell line Karpas299 xenograft model is demonstrated in WO 2014057119 A1; see, for example, FIG. 4 and the accompanying description.

### Use

The conjugates of the disclosure may be used to provide a PBD compound at a target location.

The target location is preferably a CD25+ve Acute Myeloid Leukemia (AML) cell population, such as an rAML population. The antibody is an antibody for an antigen (here, CD25) present on a AML cell population.

The target neoplasm or neoplastic cells may be all or part of a solid tumor.

“Solid tumor” herein will be understood to include solid hematological cancers such as lymphomas (Hodgkin's lymphoma or non-Hodgkin's lymphoma) which are discussed in more detail below.

The target neoplasm or neoplastic cells may be malignant.

The target neoplasm or neoplastic cells may be metastatic.

At the target location the linker may be cleaved so as to release a compound RelA, RelB, RelC, RelD or RelE. Thus, the conjugate may be used to selectively provide a compound RelA, RelB, Rel C, RelD or RelE to the target location.

The linker may be cleaved by an enzyme present at the target location.

The target location may be in vitro, in vivo or ex vivo.

The antibody-drug conjugate (ADC) compounds of the disclosure include those with utility for anticancer activity. In particular, the compounds include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

One of ordinary skill in the art is readily able to determine whether or not a candidate conjugate treats a proliferative condition for any particular cell type. For example, assays which may conveniently be used to assess the activity offered by a particular compound are described in the examples below.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis.

Any type of cell may in principal be treated, including but not limited to, lung, gastrointestinal (including, e.g. bowel, colon), breast (mammary), ovarian, prostate, liver (hepatic), kidney (renal), bladder, pancreas, brain, and skin.

Example disorders include, but are not limited to, Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL) and leukemias such as Hairy cell leukemia (HCL), Hairy cell leukemia variant (HCL-v), Acute Lymphoblastic Leukemia (ALL) such as Philadelphia chromosome-positive ALL (Ph+ALL) or Philadelphia chromosome-negative ALL (Ph-ALL) [Fielding A., Haematologica. 2010 January; 95(1): 8-12], or myeloid Leukaemia (including relapsed or refractory AML, rAML).

Hematological targets include Hodgkin's and non-Hodgkin's Lymphomas, the latter being selected from Peripheral T cell lymphoma; Cutaneous T cell lymphoma; Follicular lymphoma (FL), DLBLC, Mantle cell lymphoma (MCL) and CLL.

In the present disclosure, particularly preferred hematological targets are AML and rAML.

The antibody-drug conjugates (ADC) disclosed herein may be used to treat various diseases or disorders, e.g. characterized by the overexpression of a tumor antigen. Exemplary conditions or hyperproliferative disorders include benign or malignant tumors; leukemia, hematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Preferred examples of cancer to be treated herein are AML and rAML.

Autoimmune diseases for which the ADC compounds may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, agraft-versus-host disease (GVHD) and autoimmune endocrine disorders (such as, for example, diabetic-related autoimmune diseases such as insulin-dependent diabetes mellitus (IDDM), Addison's disease, and autoimmune thyroid disease (e.g. Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

### Methods of Treatment

The conjugates of the present disclosure may be used in a method of therapy. Also provided is a method of treatment, comprising administering to a subject in need of treatment a therapeutically-effective amount of a conjugate compound of the disclosure. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound of the disclosure may be administered alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1l, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®, PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), of atumumab (ARZERRA®, GSK), pertuzumab (PERJETA™ OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Formulations

While it is possible for the conjugate compound to be used (e.g., administered) alone, it is often preferable to present it as a composition or formulation.

In one embodiment, the composition is a pharmaceutical composition (e.g., formulation, preparation, medicament) comprising a conjugate compound, as described herein, and a pharmaceutically acceptable carrier, diluent, or excipient.

In one embodiment, the composition is a pharmaceutical composition comprising at least one conjugate compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment the active compound is administered to a human patient as a single dose of about 3 μg per kilogram body weight of the subject per day. For example 1-5 μg per kilogram body weight of the subject per day, or 2-4 μg per kilogram body weight of the subject per day.

In one embodiment the active compound is administered to a human patient as a single dose of about 6 μg per kilogram body weight of the subject per day. For example 4-8 μg per kilogram body weight of the subject per day, or 5-7 μg per kilogram body weight of the subject per day.

In one embodiment the active compound is administered to a human patient as a single dose of about 12 μg per kilogram body weight of the subject per day. For example 9-15 μg per kilogram body weight of the subject per day, or 11-13 μg per kilogram body weight of the subject per day.

In one embodiment the active compound is administered to a human patient as a single dose of about 22 μg per kilogram body weight of the subject per day. For example 16-28 μg per kilogram body weight of the subject per day, or 19-25 μg per kilogram body weight of the subject per day.

In one embodiment the active compound is administered to a human patient as a single dose of about 32 μg per kilogram body weight of the subject per day. For example 26-38 μg per kilogram body weight of the subject per day, or 29-35 μg per kilogram body weight of the subject per day.

In one embodiment the active compound is administered to a human patient as a single dose of about 50 μg per kilogram body weight of the subject per day. For example 40-60 μg per kilogram body weight of the subject per day, or 45-55 μg per kilogram body weight of the subject per day.

In one embodiment the active compound is administered to a human patient as a single dose of about 75 μg per kilogram body weight of the subject per day. For example 65-85 μg per kilogram body weight of the subject per day, or 70-80 μg per kilogram body weight of the subject per day.

In one embodiment the active compound is administered to a human patient as a single dose of about 100 μg per kilogram body weight of the subject per day. For example 90-110 μg per kilogram body weight of the subject per day, or 95-105 μg per kilogram body weight of the subject per day.

In one embodiment the active compound is administered to a human patient as a single dose of about 125 μg per kilogram body weight of the subject per day. For example 115-135 μg per kilogram body weight of the subject per day, or 120-130 μg per kilogram body weight of the subject per day.

In one embodiment the active compound is administered to a human patient as a single dose of about 150 μg per kilogram body weight of the subject per day. For example 140-160 μg per kilogram body weight of the subject per day, or 145-165 μg per kilogram body weight of the subject per day.

In one embodiment the active compound is administered to a human patient as a single dose of about 175 μg per kilogram body weight of the subject per day. For example 165-185 μg per kilogram body weight of the subject per day, or 170-180 μg per kilogram body weight of the subject per day.

In one embodiment the active compound is administered to a human patient as a single dose of about 200 μg per kilogram body weight of the subject per day. For example 190-210 μg per kilogram body weight of the subject per day, or 195-205 μg per kilogram body weight of the subject per day.

In one embodiment the active compound is administered to a human patient as a single dose of about 300 μg per kilogram body weight of the subject per day. For example 250-350 μg per kilogram body weight of the subject per day, or 280-320 μg per kilogram body weight of the subject per day.

In one embodiment the active compound is administered to a human patient as a single dose of about 400 μg per kilogram body weight of the subject per day. For example 350-450 μg per kilogram body weight of the subject per day, or 380-420 μg per kilogram body weight of the subject per day.

In one embodiment the active compound is administered to a human patient as a single dose of about 500 μg per kilogram body weight of the subject per day. For example 450-550 μg per kilogram body weight of the subject per day, or 480-520 μg per kilogram body weight of the subject per day.

In one embodiment the active compound is administered to a human patient as a single dose of about 700 μg per kilogram body weight of the subject per day. For example 650-750 μg per kilogram body weight of the subject per day, or 680-720 μg per kilogram body weight of the subject per day.

In one embodiment the active compound is administered to a human patient as a single dose of about 900 μg per kilogram body weight of the subject per day. For example 850-950 μg per kilogram body weight of the subject per day, or 880-920 μg per kilogram body weight of the subject per day.

In one embodiment the active compound is administered to a human patient as a single dose of about 1000 μg per kilogram body weight of the subject per day. For example 900-1100 μg per kilogram body weight of the subject per day, or 980-1020 μg per kilogram body weight of the subject per day.

In one embodiment, the active compound is administered to a human patient according to the following dosage regime: about 100 mg, 3 times daily. For example, 90-110 mg, 3 times daily.

In one embodiment, the active compound is administered to a human patient according to the following dosage regime: about 150 mg, 2 times daily. For example, 140-160 mg, 3 times daily.

In one embodiment, the active compound is administered to a human patient according to the following dosage regime: about 200 mg, 2 times daily. For example, 190-210 mg, 3 times daily.

However in one embodiment, the conjugate compound is administered to a human patient according to the following dosage regime: about 50 or about 75 mg, 3 or 4 times daily. For example, 40-60 or 65-85 mg, 3 or 4 times daily.

In one embodiment, the conjugate compound is administered to a human patient according to the following dosage regime: about 100 or about 125 mg, 2 times daily. For example, 90-110 or 115-135 mg, 3 or 4 times daily.

The dosage amounts described above may apply to the conjugate (including the PBD moiety and the linker to the antibody) or to the effective amount of PBD compound provided, for example the amount of compound that is releasable after cleavage of the linker.

For the prevention or treatment of disease, the appropriate dosage of an ADC of the disclosure will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of ADC to be administered to a patient is in the range of about 0.01 to about 10 mg/kg of patient weight. For repeated administrations over several days or longer, depending on the condition, the treatment is sustained until a desired suppression of disease symptoms occurs. An exemplary dosing regimen comprises a course of administering an initial loading dose of about 3-300 μg/kg, followed by additional doses every week, two weeks, or three weeks of an ADC. Other dosage regimens may be useful. The progress of this therapy is easily monitored by conventional techniques and assays.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

### Degree of Subject/Patient Response

A subject/patient may respond to treatment with a complete response (CR), CR with incomplete blood count recovery (CRi), partial response (PR), or no response (NR).

Complete response (CR) is defined as achieving each of the following:
Bone marrow differential showing ≤5% blast cells and absence of blast cells with Auer rods.
Absolute neutrophil count (ANC) ≥1.0×10⁹/L and platelet count ≥100×10⁹/L.
Absence of extra-medullary disease.
Patient is independent of red blood cell (RBC) transfusions.

Complete response with incomplete blood count recovery (CRi) is defined as achieving all CR criteria except that values for ANC may be <1.0×10⁹/L and/or values for platelets may be <100×10⁹/L.

Partial response (PR) is defined as achieving the following:
ANC ≥1.0×109/L and platelet count ≥100×109/L.
Bone marrow differential showing a ≥50% decrease from baseline in the percentage of bone marrow blast cells to a level >5% and ≤25%, or
bone marrow differential showing <5% blast cells and presence of Auer rods.

No response (NR) is defined as not achieving CR, CRi, or PR.

Assessment of response to treatment can be based on a bone marrow aspirate (or biopsy if aspirate unattainable). In an example assessment, the first bone marrow sample is obtained 14 (±3) days after the first dose and is repeated 14 (±3) days after each subsequent dose, until disease progression or complete response (CR) or CR with incomplete blood count recovery (CRi) is achieved; once CR/CRi is achieved, sampling is repeated as clinically required.

Accordingly, in some embodiments at least 20% of subjects/patients achieve CR or CRi following administration of one or more doses of the conjugates described herein. The subjects/patients may be AML or rAML patients. Preferably, at least 30% of subjects/patients achieve CR or CRi following administration of one or more doses of the conjugates described herein, such as at least 40%, at least 50%, at least 60%, at least 70%, at least 80%, at least 90%, at least 95%, at least 98%, or at least 99% of subjects/patients. The number of doses required to achieve CR or CRi may be two, three, four, five, ten or more. In some embodiments the patients achieve CR or CRi no more than a year after administration of the first dose, such as no more than 6 months, no more than 3 months, no more than a month, no more than a fortnight, or no more than a week after administration of the first dose.

### The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human.

### Further Preferences

The following preferences may apply to all aspects of the disclosure as described above, or may relate to a single aspect. The preferences may be combined together in any combination.

In some embodiments, R^{6′}, R^{7′}, R^{9′}, and Y′ are preferably the same as R⁶, R⁷, R⁹, and Y respectively.

### Dimer Link

Y and Y′ are preferably O.

R″ is preferably a C₃₋₇ alkylene group with no substituents. More preferably R″ is a C₃, C₅ or C₇ alkylene. Most preferably, R″ is a C₃ or C₅ alkylene.

R⁶ to R⁹

R⁹ is preferably H.

R⁶ is preferably selected from H, OH, OR, SH, NH₂, nitro and halo, and is more preferably H or halo, and most preferably is H.

R⁷ is preferably selected from H, OH, OR, SH, SR, NH₂, NHR, NRR′, and halo, and more preferably independently selected from H, OH and OR, where R is preferably selected from optionally substituted C₁₋₇ alkyl, C₃₋₁₀ heterocyclyl and C₅₋₁₀ aryl groups. R may be more preferably a C₁₋₄ alkyl group, which may or may not be substituted. A substituent of interest is a C₅₋₆ aryl group (e.g. phenyl). Particularly preferred substituents at the 7-positions are OMe and OCH₂Ph. Other substituents of particular interest are dimethylamino (i.e. —NMe₂); —(OC₂H₄)_{q}OMe, where q is from 0 to 2; nitrogen-containing C heterocyclyls, including morpholino, piperidinyl and N-methyl-piperazinyl.

These preferences apply to R^{9′}, R^{6′} and R^{7′} respectively.

### R¹²

When there is a double bond present between C2′ and C3′, R¹² is selected from:

(a) C₅₋₁₀ aryl group, optionally substituted by one or more substituents selected from the group comprising: halo, nitro, cyano, ether, C₁₋₇ alkyl, C₃₋₇ heterocyclyl and bis-oxy-C₁₋₃ alkylene;
(b) C₁₋₅ saturated aliphatic alkyl;
(c) C₃₋₆ saturated cycloalkyl;

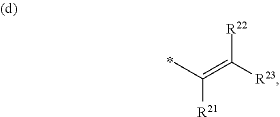

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

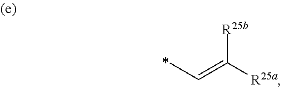

wherein one of R^{25a} and R^{25b} is H and the other is selected from: phenyl, which phenyl is optionally substituted by a group selected from halo methyl, methoxy; pyridyl; and thiophenyl; and

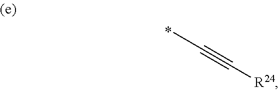

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl.

When R¹² is a C₅₋₁₀ aryl group, it may be a C₅₋₇ aryl group. A C₅₋₇ aryl group may be a phenyl group or a C₅₋₇ heteroaryl group, for example furanyl, thiophenyl and pyridyl. In some embodiments, R¹² is preferably phenyl. In other embodiments, R¹² is preferably thiophenyl, for example, thiophen-2-yl and thiophen-3-yl.

When R¹² is a C₅₋₁₀ aryl group, it may be a C₈₋₁₀ aryl, for example a quinolinyl or isoquinolinyl group. The quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. Of these quinolin-3-yl and quinolin-6-yl may be preferred. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. Of these isoquinolin-3-yl and isoquinolin-6-yl may be preferred.

When R¹² is a C₅₋₁₀ aryl group, it may bear any number of substituent groups. It preferably bears from 1 to 3 substituent groups, with 1 and 2 being more preferred, and singly substituted groups being most preferred. The substituents may be any position.

Where R¹² is C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

Where R¹² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

R¹² Substituents, when R¹² is a C₅₋₁₀ Aryl Group

If a substituent on R¹² when R¹² is a C₅₋₁₀ aryl group is halo, it is preferably F or Cl, more preferably Cl.

If a substituent on R¹² when R¹² is a C₅₋₁₀ aryl group is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy). The alkoxy group may itself be further substituted, for example by an amino group (e.g. dimethylamino).

If a substituent on R¹² when R¹² is a C₅₋₁₀ aryl group is C₁₋₇ alkyl, it may preferably be a C₁₋₄ alkyl group (e.g. methyl, ethyl, propryl, butyl).

If a substituent on R¹² when R¹² is a C₅₋₁₀ aryl group is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups. If the C₆ nitrogen containing heterocyclyl group is piperazinyl, the said further substituent may be on the second nitrogen ring atom.

If a substituent on R¹² when R¹² is a C₅₋₁₀ aryl group is bis-oxy-C₁₋₃ alkylene, this is preferably bis-oxy-methylene or bis-oxy-ethylene.

If a substituent on R¹² when R¹² is a C₅₋₁₀ aryl group is ester, this is preferably methyl ester or ethyl ester.

Particularly preferred substituents when R¹² is a C₅₋₁₀ aryl group include methoxy, ethoxy, fluoro, chloro, cyano, bis-oxy-methylene, methyl-piperazinyl, morpholino and methylthiophenyl. Other particularly preferred substituent for R¹² are dimethylaminopropyloxy and carboxy.

Particularly preferred substituted R¹² groups when R¹² is a C₅₋₁₀ aryl group include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthiophenyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl. Another possible substituted R¹² group is 4-nitrophenyl. R¹² groups of particular interest include 4-(4-methylpiperazin-1-yl)phenyl and 3,4-bisoxymethylene-phenyl.

When R¹² is C₁₋₅ saturated aliphatic alkyl, it may be methyl, ethyl, propyl, butyl or pentyl. In some embodiments, it may be methyl, ethyl or propyl (n-pentyl or isopropyl). In some of these embodiments, it may be methyl. In other embodiments, it may be butyl or pentyl, which may be linear or branched.

When R¹² is C₃₋₆ saturated cycloalkyl, it may be cyclopropyl, cyclobutyl, cyclopentyl or cyclohexyl. In some embodiments, it may be cyclopropyl.

When R¹² is

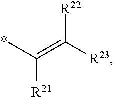

each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5. In some embodiments, the total number of carbon atoms in the R¹² group is no more than 4 or no more than 3.

In some embodiments, one of R²¹, R²² and R²³ is H, with the other two groups being selected from H, C₁₋₃ saturated alkyl, C₂₋₃ alkenyl, C₂₋₃ alkynyl and cyclopropyl.

In other embodiments, two of R²¹, R²² and R²³ are H, with the other group being selected from H, C₁₋₃ saturated alkyl, C₂₋₃ alkenyl, C₂₋₃ alkynyl and cyclopropyl.

In some embodiments, the groups that are not H are selected from methyl and ethyl. In some of these embodiments, the groups that re not H are methyl.

In some embodiments, R²¹ is H.

In some embodiments, R²² is H.

In some embodiments, R²³ is H.

In some embodiments, R²¹ and R²² are H.

In some embodiments, R²¹ and R²³ are H.

In some embodiments, R²² and R²³ are H.

An R¹² group of particular interest is:

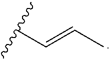 [SMILES: */C=C/C]

When R¹² is

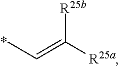

one of R^{25a} and R^{25b} is H and the other is selected from: phenyl, which phenyl is optionally substituted by a group selected from halo, methyl, methoxy; pyridyl; and thiophenyl. In some embodiments, the group which is not H is optionally substituted phenyl. If the phenyl optional substituent is halo, it is preferably fluoro. In some embodiment, the phenyl group is unsubstituted.

When R¹² is

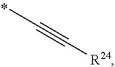

R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl. If the phenyl optional substituent is halo, it is preferably fluoro. In some embodiment, the phenyl group is unsubstituted.

In some embodiments, R²⁴ is selected from H, methyl, ethyl, ethenyl and ethynyl. In some of these embodiments, R²⁴ is selected from H and methyl.

When there is a single bond present between C2′ and C3′,

R²

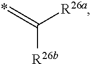

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester.

In some embodiments, it is preferred that R^{26a} and R^{26b} are both H.

In other embodiments, it is preferred that R^{26a} and R^{26b} are both methyl.

In further embodiments, it is preferred that one of R^{26a} and R^{26b} is H, and the other is selected from C₁₋₄ saturated alkyl, C₂₋₃ alkenyl, which alkyl and alkenyl groups are optionally substituted. In these further embodiment, it may be further preferred that the group which is not H is selected from methyl and ethyl.

### R²

The above preferences for R¹² apply equally to R².

### R²²

In some embodiments, R²² is of formula IIa.

A in R²² when it is of formula Ia may be phenyl group or a C₅₋₇ heteroaryl group, for example furanyl, thiophenyl and pyridyl. In some embodiments, A is preferably phenyl.

Q²-X may be on any of the available ring atoms of the C₅₋₇ aryl group, but is preferably on a ring atom that is not adjacent the bond to the remainder of the compound, i.e. it is preferably β or γ to the bond to the remainder of the compound. Therefore, where the C₅₋₇ aryl group (A) is phenyl, the substituent (Q²-X) is preferably in the meta- or para-positions, and more preferably is in the para-position.

In some embodiments, Q¹ is a single bond. In these embodiments, Q² is selected from a single bond and —Z—(CH₂)ₙ—, where Z is selected from a single bond, O, S and NH and is from 1 to 3. In some of these embodiments, Q² is a single bond. In other embodiments, Q² is —Z—(CH₂)ₙ—. In these embodiments, Z may be O or S and n may be 1 or n may be 2. In other of these embodiments, Z may be a single bond and n may be 1.

In other embodiments, Q¹ is —CH═CH—.

In other embodiments, R²² is of formula IIb. In these embodiments, R^{C1}, R^{C2} and R^{C3} are independently selected from H and unsubstituted C₁₋₂ alkyl. In some preferred embodiments, R^{C1}, R^{C2} and R^{C3} are all H. In other embodiments, R^{C1}, R^{C2} and R^{C3} are all methyl. In certain embodiments, R^{C1}, R^{C2} and R^{C3} are independently selected from H and methyl.

X is a group selected from the list comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

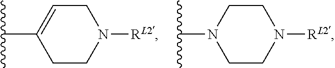

NR^{N}R^{L2′}, wherein R^{N} is selected from the group comprising H and C₁₋₄ alkyl. X may preferably be: OH, SH, CO₂H, —N═C═O or NHR^{N}, and may more preferably be: O—R^{L2′}, S—R^{L2′}, CO₂—R^{L2′}, —NH—C(═O)—R^{L2′} or NH—R^{L2′}. Particularly preferred groups include: O—R^{L2′}, S—R^{L2′} and NH—R^{L2′}, with NH—R^{L2′} being the most preferred group.

In some embodiments R²² is of formula IIc. In these embodiments, it is preferred that Q is NR^{N}—R^{L2′}. In other embodiments, Q is O—R^{L2′}. In further embodiments, Q is S—R^{L2′}. R^{N} is preferably selected from H and methyl. In some embodiment, R^{N} is H. In other embodiments, R^{N} is methyl.

In some embodiments, R²² may be -A-CH₂—X and -A-X. In these embodiments, X may be O—R^{L2′}, S—R^{L2′}, CO₂—R^{L2′}, CO—R^{L2′} and NH—R^{L2′}. In particularly preferred embodiments, X may be NH—R^{L2′}.

R¹⁰, R¹¹

In some embodiments, R¹⁰ and R¹¹ together form a double bond between the nitrogen and carbon atoms to which they are bound.

In some embodiments, R¹¹ is OH.

In some embodiments, R¹¹ is OMe.

In some embodiments, R¹¹ is SO_{z}M, where z is 2 or 3 and M is a monovalent pharmaceutically acceptable cation.

### R^{11a}

In some embodiments, R^{11a} is OH.

In some embodiments, R^{11a} is OMe.

In some embodiments, R^{11a} is SO_{z}M, where z is 2 or 3 and M is a monovalent pharmaceutically acceptable cation.

R²⁰, R²¹

In some embodiments, R²⁰ and R²¹ together form a double bond between the nitrogen and carbon atoms to which they are bound.

In some embodiments R²⁰ is H.

In some embodiments, R²⁰ is R^{C}.

In some embodiments, R²¹ is OH.

In some embodiments, R²¹ is OMe.

In some embodiments, R²¹ is SO_{z}M, where z is 2 or 3 and M is a monovalent pharmaceutically acceptable cation.

R³⁰, R³¹

In some embodiments, R³⁰ and R³¹ together form a double bond between the nitrogen and carbon atoms to which they are bound.

In some embodiments, R³¹ is OH.

In some embodiments, R³¹ is OMe.

In some embodiments, R³¹ is SO_{z}M, where z is 2 or 3 and M is a monovalent pharmaceutically acceptable cation.

### M and z

It is preferred that M is a monovalent pharmaceutically acceptable cation, and is more preferably Na⁺.

z is preferably 3.

Preferred conjugates of the first aspect of the present disclosure may have a D^{L} of formula Ia:

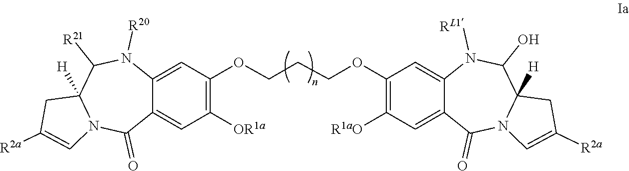

where
R^{L1′}, R²⁰ and R²¹ are as defined above;
n is 1 or 3;
R^{1a} is methyl or phenyl; and
R^{2a} is selected from:

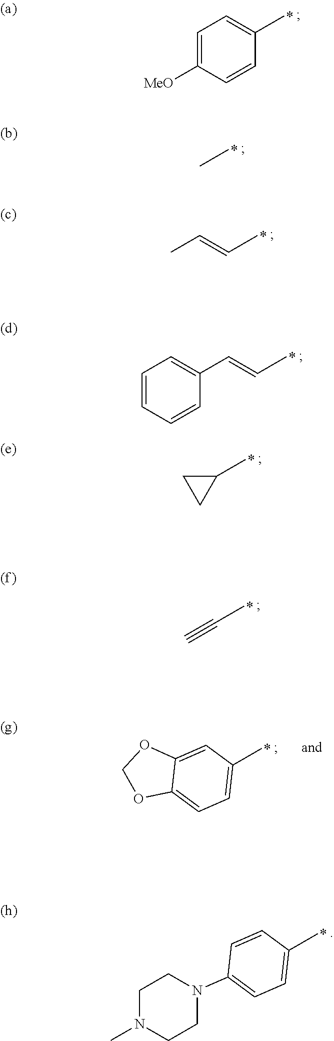

Preferred conjugates of the first aspect of the present disclosure may have a D^{L} of formula Ib:

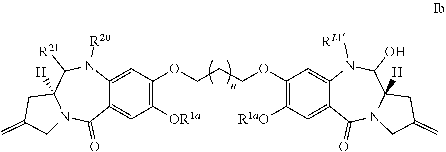

where
R^{L1′}, R²⁰ and R²¹ are as defined above;
n is 1 or 3; and
R^{1a} is methyl or phenyl.

Preferred conjugates of the first aspect of the present disclosure may have a D^{L} of formula Ic:

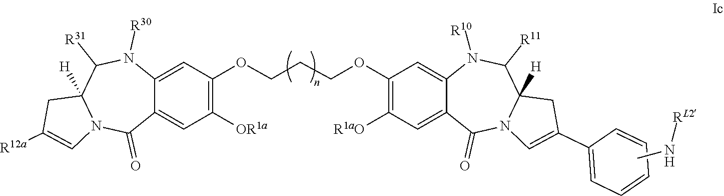

where R^{L2′}, R¹⁰, R¹¹, R³⁰ and R³¹ are as defined above
n is 1 or 3;
R^{12a} is selected from:

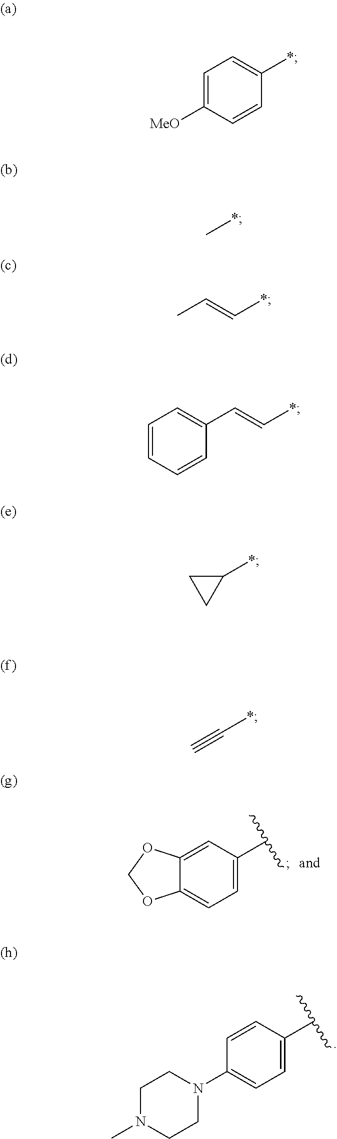

the amino group is at either the meta or para positions of the phenyl group.

Preferred conjugates of the first aspect of the present disclosure may have a D^{L} of formula Id:

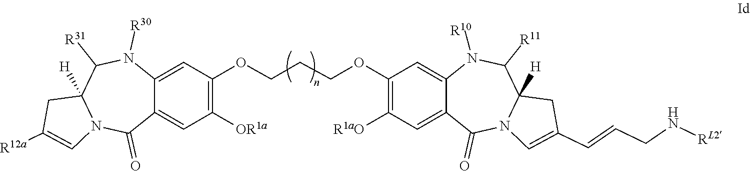

where R^{L2′}, R¹⁰, R¹¹, R³⁰ and R³¹ are as defined above
n is 1 or 3;
R^{1a} is methyl or phenyl;
R^{12a} is selected from:

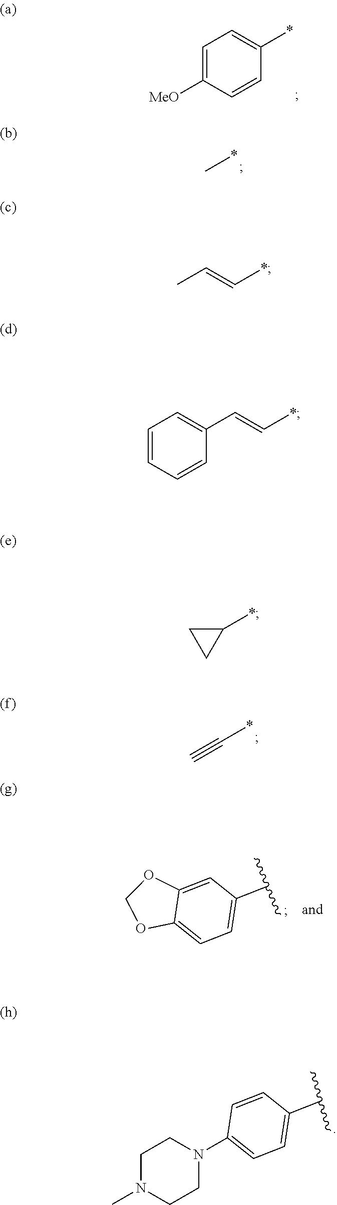

Preferred conjugates of the first aspect of the present disclosure may have a D^{L} of formula Ie:

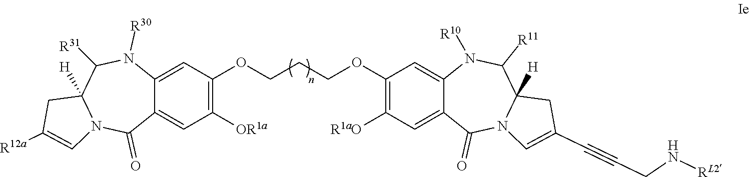

where R^{L2′}, R¹⁰, R¹¹, R³⁰ and R³¹ are as defined above
n is 1 or 3;
R^{1a} is methyl or phenyl;
R^{12a} is selected from:

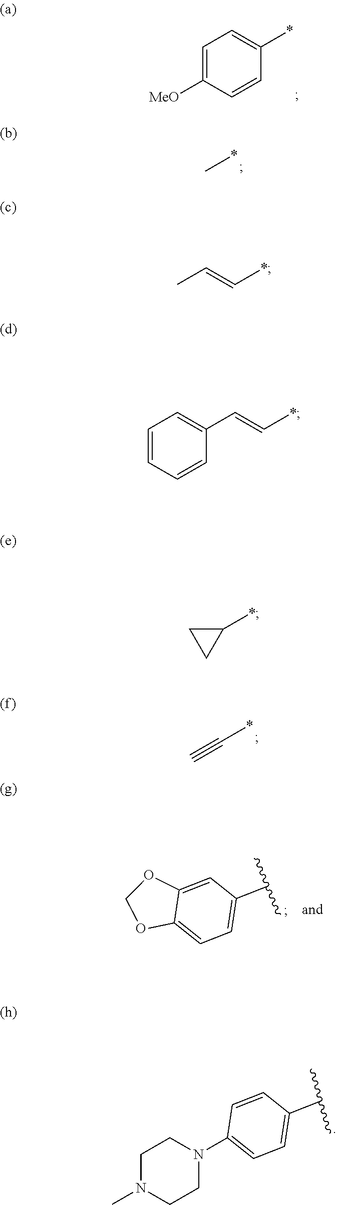

### Some Embodiments

1. A method of treating CD25+ve Acute Myeloid Leukemia (AML), in a subject, said method comprising administering to a subject a conjugate of formula L-(D^{L})ₚ, where D^{L} is of formula I or II:

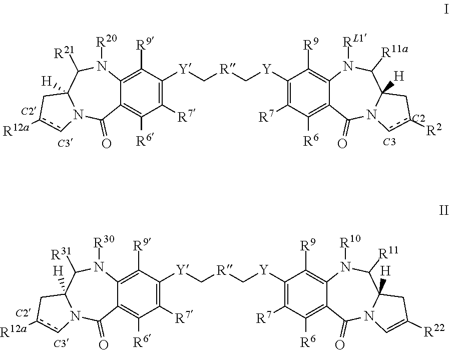

wherein:
L is an antibody (Ab) which is an antibody that binds to CD25;
when there is a double bond present between C2′ and C3′, R¹² is selected from the group consisting of:
   (ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
   (ib) C₁₋₅ saturated aliphatic alkyl;
   (ic) C₃₋₆ saturated cycloalkyl;

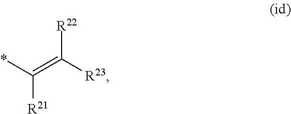

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

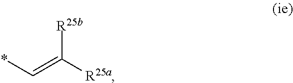

wherein one of R^{25a} and R^{25b} is H and the other is selected from: phenyl, which phenyl is optionally substituted by a group selected from halo, methyl, methoxy; pyridyl; and thiophenyl; and

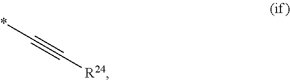

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

R¹² is

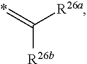

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;

### [Formula I]

R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

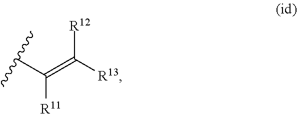

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

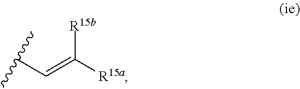

wherein one of R^{15a} and R^{15b} is H and the other is selected from: phenyl, which phenyl is optionally substituted by a group selected from halo, methyl, methoxy; pyridyl; and thiophenyl; and

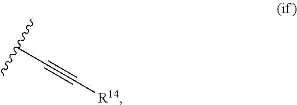

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

R² is

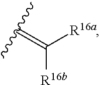

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula IIIa, formula IIIb or formula IIIc:

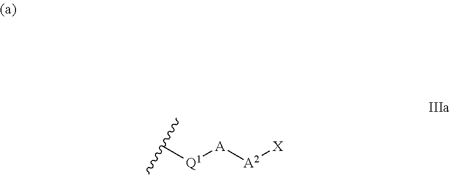

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

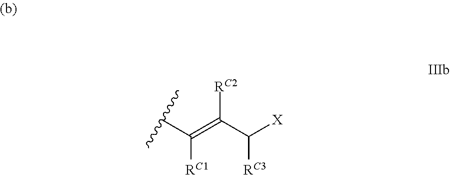

where;
R^{C1}, R^{C2} and R^{C3} are independently selected from H and unsubstituted C₁₋₂ alkyl;

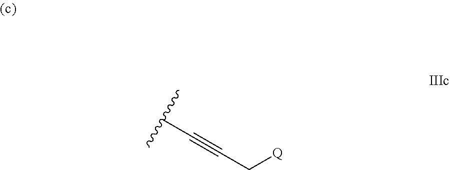

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

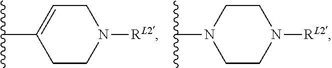

NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M;
optionally wherein the CD25+ve Acute Myeloid Leukemia comprises both CD25+ve and CD25-ve cells.
   2. The method according to paragraph 1, wherein the CD25+ve Acute Myeloid Leukemia (AML) is:
(i) refractory AML; or
(ii) relapsed AML.
   3. The method according to either paragraph 1 or paragraph 2, wherein R⁷ is selected from H, OH and OR.
   4. The method according to paragraph 3, wherein R⁷ is a C₁₋₄ alkyloxy group.
   5. The method according to any one of paragraphs 1 to 4, wherein Y is O.
   6. The method according to any one of the preceding paragraphs, wherein R″ is C₃₋₇ alkylene.
   7. The method according to any one of paragraphs 1 to 6, wherein R⁹ is H.
   8. The method according to any one of paragraphs 1 to 7, wherein R⁶ is selected from H and halo.
   9. The method according to any one of paragraphs 1 to 8, wherein there is a double bond between C2′ and C3′, and R¹² is a C₅₋₇ aryl group.
   10. The method according to paragraph 9, wherein R¹² is phenyl.
   11. The method according to any one of paragraphs 1 to 8, wherein there is a double bond between C2′ and C3′, and R¹² is a C₈₋₁₀ aryl group.
   12. The method according to any one of paragraphs 9 to 11, wherein R¹² bears one to three substituent groups.
   13. The method according to any one of paragraphs 9 to 12, wherein the substituents are selected from methoxy, ethoxy, fluoro, chloro, cyano, bis-oxy-methylene, methyl-piperazinyl, morpholino and methyl-thiophenyl.
   14. The method according to any one of paragraphs 1 to 8, wherein there is a double bond between C2′ and C3′, and R¹² is a C₁₋₅ saturated aliphatic alkyl group.
   15. The method according to paragraph 14, wherein R¹² is methyl, ethyl or propyl.
   16. The method according to any one of paragraphs 1 to 8, wherein there is a double bond between C2′ and C3′, and R¹² is a C₃₋₆ saturated cycloalkyl group.
   17. The method according to paragraph 16, wherein R¹² is cyclopropyl.
   18. The method according to any one of paragraphs 1 to 8, wherein there is a double bond between C2′ and C3′, and R¹² is a group of formula:

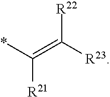

19. The method according to paragraph 18, wherein the total number of carbon atoms in the R¹² group is no more than 4.
20. The method according to paragraph 19, wherein the total number of carbon atoms in the R¹² group is no more than 3.

21. The method according to any one of paragraphs 18 to 20, wherein one of R²¹, R²² and R²³ is H, with the other two groups being selected from H, C₁₋₃ saturated alkyl, C₂₋₃ alkenyl, C₂₋₃ alkynyl and cyclopropyl.

22. The method according to any one of paragraphs 18 to 20, wherein two of R²¹, R²² and R²³ are H, with the other group being selected from H, C₁₋₃ saturated alkyl, C₂₋₃ alkenyl, C₂₋₃ alkynyl and cyclopropyl.
23. The method according to any one of paragraphs 1 to 8, wherein there is a double bond between C2′ and C3′, and R¹² is a group of formula:

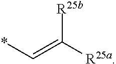

24. The method according to paragraph 23, wherein R¹² is the group:

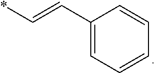 [SMILES: */C=C/c1ccccc1]

25. The method according to any one of paragraphs 1 to 8, wherein there is a double bond between C2′ and C3′, and R¹² is a group of formula:

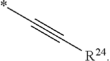

26. The method according to paragraph 25, wherein R²⁴ is selected from H, methyl, ethyl, ethenyl and ethynyl.
27. The method according to paragraph 26, wherein R²⁴ is selected from H and methyl.
28. The method according to any one of paragraphs 1 to 8, wherein there is a single bond between C2′ and C3′, R¹² is

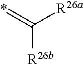

and R^{26a} and R^{26b} are both H.
29. The method according to any one of paragraphs 1 to 8, wherein there is a single bond between C2′ and C3′, R¹² is

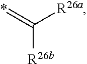

and R^{26a} and R^{26b} are both methyl.
30. The method according to any one of paragraphs 1 to 8, wherein there is a single bond between C2′ and C3′, R¹² is

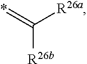

one of R^{26a} and R^{26b} is H, and the other is selected from C₁₋₄ saturated alkyl, C₂₋₃ alkenyl, which alkyl and alkenyl groups are optionally substituted.

### [Formula I]

31. The method according to any one of paragraphs 1 to 30, wherein there is a double bond between C2 and C3, and R² is a C₅₋₇ aryl group.
32. The method according to paragraph 31, wherein R² is phenyl.
33. The method according to any one of paragraphs 1 to 30, wherein there is a double bond between C2 and C3, and R¹ is a C₈₋₁₀ aryl group.
34. A compound according to any one of paragraphs 31 to 33, wherein R² bears one to three substituent groups.
35. The method according to any one of paragraphs 31 to 34, wherein the substituents are selected from methoxy, ethoxy, fluoro, chloro, cyano, bis-oxy-methylene, methyl-piperazinyl, morpholino and methyl-thiophenyl.
36. The method according to any one of paragraphs 1 to 30, wherein there is a double bond between C2 and C3, and R² is a C₁₋₅ saturated aliphatic alkyl group.
37. The method according to paragraph 36, wherein R² is methyl, ethyl or propyl.
38. The method according to any one of paragraphs 1 to 30, wherein there is a double bond between C2 and C3, and R² is a C₃₋₆ saturated cycloalkyl group.
39. The method according to paragraph 38, wherein R² is cyclopropyl.
40. The method according to any one of paragraphs 1 to 30, wherein there is a double bond between C2 and C3, and R² is a group of formula:

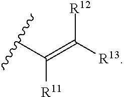

41. The method according to paragraph 40, wherein the total number of carbon atoms in the R² group is no more than 4.
42. The method according to paragraph 41, wherein the total number of carbon atoms in the R² group is no more than 3.
43. The method according to any one of paragraphs 40 to 42, wherein one of R¹¹, R¹² and R¹³ is H, with the other two groups being selected from H, C₁₋₃ saturated alkyl, C₂₋₃ alkenyl, C₂₋₃ alkynyl and cyclopropyl.
44. The method according to any one of paragraphs 40 to 42, wherein two of R¹¹, R¹² and R¹³ are H, with the other group being selected from H, C₁₋₃ saturated alkyl, C₂₋₃ alkenyl, C₂₋₃ alkynyl and cyclopropyl.
45. The method according to any one of paragraphs 1 to 30, wherein there is a double bond between C2 and C3, and R² is a group of formula:

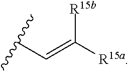

46. The method according to paragraph 45, wherein R² is the group:

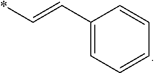 [SMILES: */C=C/c1ccccc1]

47. The method according to any one of paragraphs 1 to 30, wherein there is a double bond between C2 and C3, and R² is a group of formula:

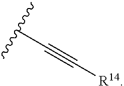

48. The method according to paragraph 47, wherein R¹⁴ is selected from H, methyl, ethyl, ethenyl and ethynyl.
49. The method according to paragraph 48, wherein R¹⁴ is selected from H and methyl.
50. The method according to any one of paragraphs 1 to 30, wherein there is a single bond between C2 and C3, R² is

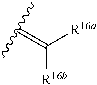

and R^{16a} and R^{16b} are both H.
51. The method according to any one of paragraphs 1 to 30, wherein there is a single bond between C2 and C3, R² is

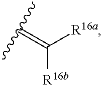

and R^{16a} and R^{16b} are both methyl.
52. The method according to any one of paragraphs 1 to 30, wherein there is a single bond between C2 and C3, R² is

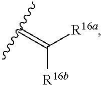

one of R^{16a} and R^{16b} is H, and the other is selected from C₁₋₄ saturated alkyl, C₂₋₃ alkenyl, which alkyl and alkenyl groups are optionally substituted.
53. The method according to any one of paragraphs 1 to 52, wherein R¹¹ is OH.
54. The method according to any one of paragraphs 1 to 53, wherein R²¹ is OH.
55. The method according to any one of paragraphs 1 to 53, wherein R²¹ is OMe.
56. The method according to any one of paragraphs 1 to 55, wherein R²⁰ is H.
57. The method according to any one of paragraphs 1 to 55, wherein R²⁰ is R^{C}.
58. The method according to paragraph 57, wherein R^{C} is selected from the group consisting of: Alloc, Fmoc, Boc, Troc, Teoc, Psec, Cbz and PNZ.
60. The method according to paragraph 57, wherein R^{C} is a group:

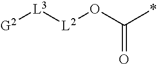

where the asterisk indicates the point of attachment to the N10 position, G² is a terminating group, L³ is a covalent bond or a cleavable linker L¹, L² is a covalent bond or together with OC(═O) forms a self-immolative linker.
   61. The method according to paragraph 60, wherein G² is Ac or Moc or is selected from the group consisting of: Alloc, Fmoc, Boc, Troc, Teoc, Psec, Cbz and PNZ.
   62. The method according to any one of paragraphs 1 to 53, wherein R²⁰ and R²¹ together form a double bond between the nitrogen and carbon atoms to which they are bound.

### [Formula II]

63. The method according to any one of paragraphs 1 to 30, wherein R²² is of formula IIIa, and A is phenyl.
64. The method according to any one of paragraphs 1 to 30 and paragraph 63, wherein R²² is of formula IIa, and Q¹ is a single bond.
65. The method according to paragraph 63, wherein Q² is a single bond.
66. The method according to paragraph 63, wherein Q² is —Z—(CH₂)ₙ—, Z is O or S and n is 1 or 2.
67. The method according any one of paragraphs 1 to 30 and paragraph 63, wherein R²² is of formula IIIa, and Q¹ is —CH═CH—.
68. The method according to any one of paragraphs 1 to 30, wherein R²² is of formula IIIb,
and R^{C1}, R^{C2} and R^{C3} are independently selected from H and methyl.
69. The method according to paragraph 68, wherein R^{C1}, R^{C2} and R^{C3} are all H.
70. The method according to paragraph 68, wherein R^{C1}, R^{C2} and R^{C3} are all methyl.
71. The method according to any one of paragraphs 1 to 30 and paragraphs 63 to 70, wherein R²² is of formula IIIa or formula IIb and X is selected from O—R^{L2′}, S—R^{L2′}, CO₂—R^{L2′}, —N—C(═O)—R^{L2′} and NH—R^{L2′}.
72. The method according to paragraph 71, wherein X is NH—R^{L2′}.
73. The method according to any one of paragraphs 1 to 30, wherein R²² is of formula IIIc, and Q is NR^{N}—R^{L2′}.
74. The method according to paragraph 73, wherein R^{N} is H or methyl.
75. The method according to any one of paragraphs 1 to 30, wherein R²² is of formula IIIc, and Q is O—R^{L2′} or S—R^{L2′}.
76. The method according to any one of paragraphs 1 to 30 and paragraphs 63 to 75, wherein R¹¹ is OH.
77. The method according to any one of paragraphs 1 to 30 and paragraphs 63 to 75, wherein R¹¹ is OMe.
78. The method according to any one of paragraphs 1 to 30 and paragraphs 63 to 77, wherein R¹⁰ is H.
79. The method according to any one of paragraphs 1 to 30 and paragraphs 63 to 75, wherein R¹⁰ and R¹¹ together form a double bond between the nitrogen and carbon atoms to which they are bound.
80. The method according to any one of paragraphs 1 to 30 and paragraphs 63 to 79, wherein R³¹ is OH.
81. The method according to any one of paragraphs 1 to 30 and paragraphs 63 to 79, wherein R³¹ is OMe.
82. The method according to any one of paragraphs 1 to 30 and paragraphs 63 to 81, wherein R³⁰ is H.
83. The method according to any one of paragraphs 1 to 30 and paragraphs 63 to 79, wherein R³⁰ and R³¹ together form a double bond between the nitrogen and carbon atoms to which they are bound.
84. The method according to any one of paragraphs 1 to 83, wherein R^{6′}, R^{7′}, R^{9′}, and Y′ are the same as R⁶, R⁷, R⁹, and Y.
85. The method according to any one of paragraphs 1 to 84 wherein, wherein L-R^{L1′} or L-R^{L2′} is a group:

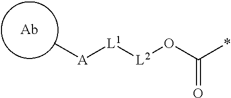

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.
   86. The method of paragraph 85, wherein L¹ is enzyme cleavable.
   87. The method of paragraph 85 or paragraph 86, wherein L¹ comprises a contiguous sequence of amino acids.
   88. The method of paragraph 87, wherein L¹ comprises a dipeptide and the group —X₁—X₂— in dipeptide, —NH—X₁—X₂—CO—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-,
-Trp-Cit-.
   89. The method according to paragraph 88, wherein the group —X₁—X₂— in dipeptide, —NH—X₁—X₂—CO—, is selected from:
-Phe-Lys-,
-Val-Ala-,
-Val-Lys-,
-Ala-Lys-,
-Val-Cit-.
   90. The method according to paragraph 89, wherein the group —X₁—X₂— in dipeptide, —NH—X₁—X₂—CO—, is -Phe-Lys-, -Val-Ala- or -Val-Cit-.
   91. The method according to any one of paragraphs 88 to 90, wherein the group X₂—CO— is connected to L².
   92. The method according to any one of paragraphs 88 to 91, wherein the group NH—X₁— is connected to A.
   93. The method according to any one of paragraphs 88 to 92, wherein L² together with OC(═O) forms a self-immolative linker.
   94. The method according to paragraph 93, wherein C(═O)O and L² together form the group:

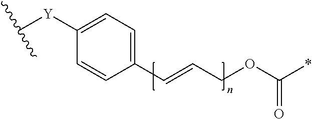

where the asterisk indicates the point of attachment to the PBD, the wavy line indicates the point of attachment to the linker L¹, Y is NH, O, C(═O)NH or C(═O)O, and n is 0 to 3.
   95. The method according to paragraph 94, wherein Y is NH.
   96. The method according to paragraph 94 or paragraph 95, wherein n is 0.
   97. The method according to paragraph 95, wherein L¹ and L² together with —OC(═O)— comprise a group selected from:

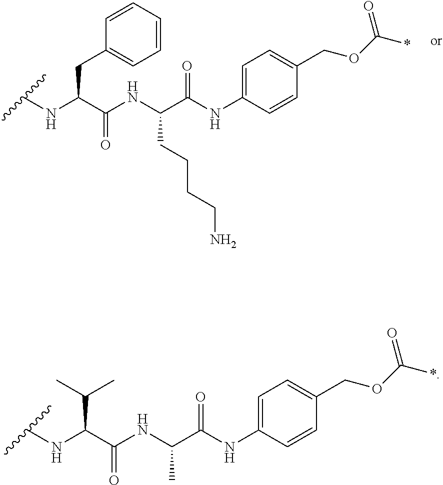

where the asterisk indicates the point of attachment to the PBD, and the wavy line indicates the point of attachment to the remaining portion of the linker L¹ or the point of attachment to A.
   98. The method according to paragraph 97, wherein the wavy line indicates the point of attachment to A.
   99. The method according to any one of paragraphs 85 to 98, wherein A is:

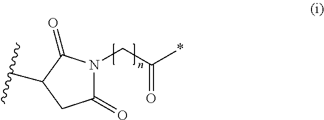

where the asterisk indicates the point of attachment to L¹, the wavy line indicates the point of attachment to the antibody, and n is 0 to 6; or

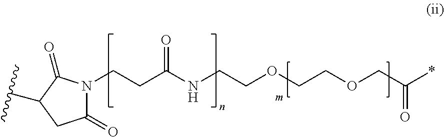

where the asterisk indicates the point of attachment to L¹, the wavy line indicates the point of attachment to the antibody, n is 0 or 1, and m is 0 to 30.
   100. A method according to paragraph 1 wherein
   D^{L} is selected from the group comprising:

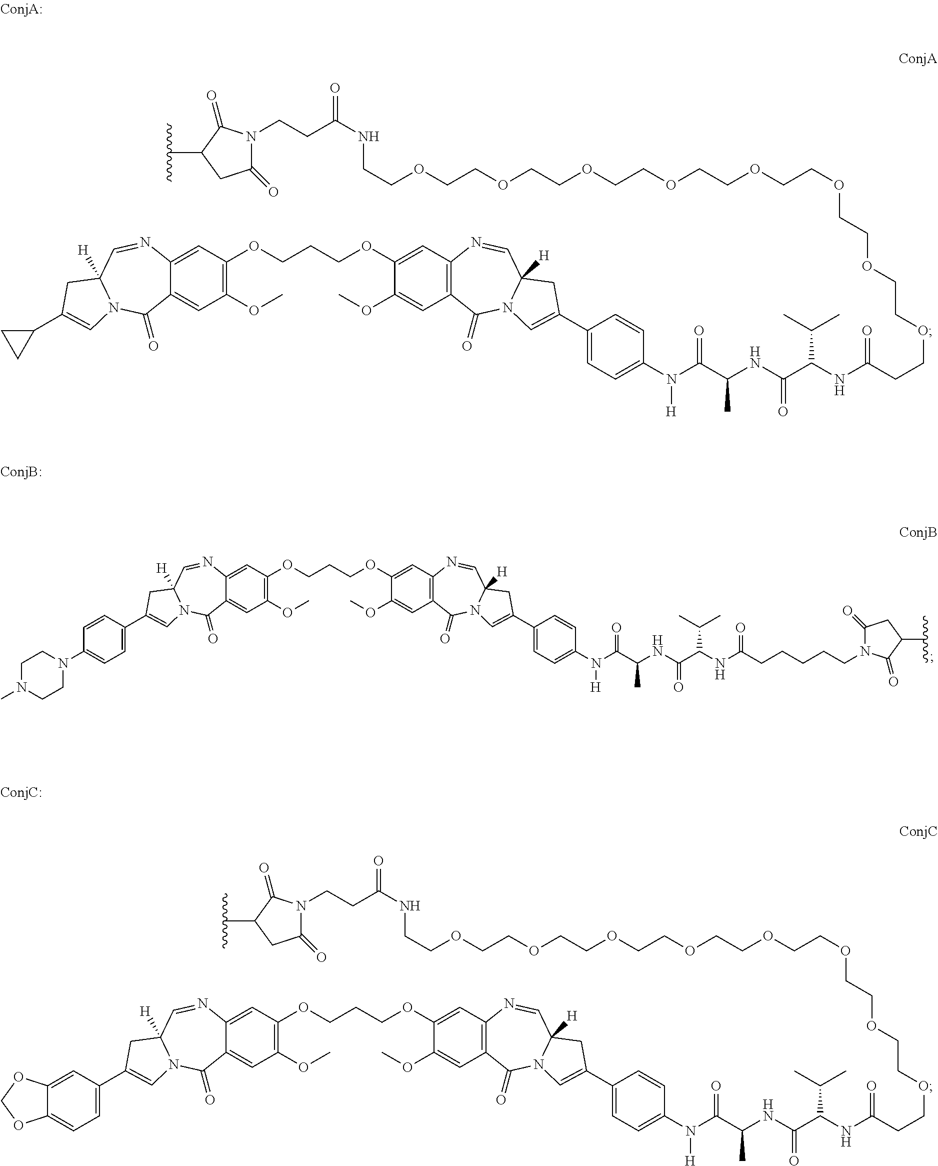

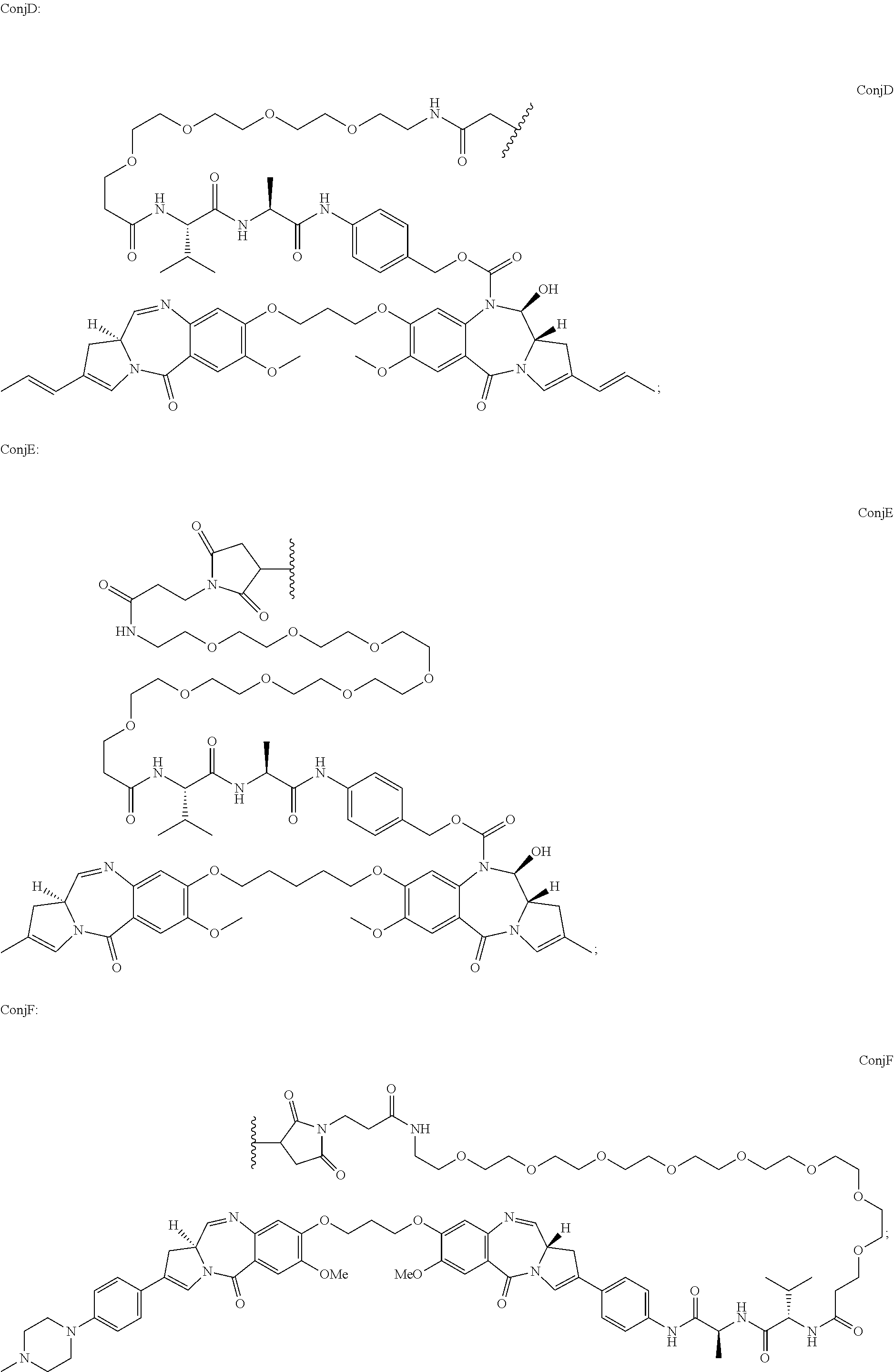

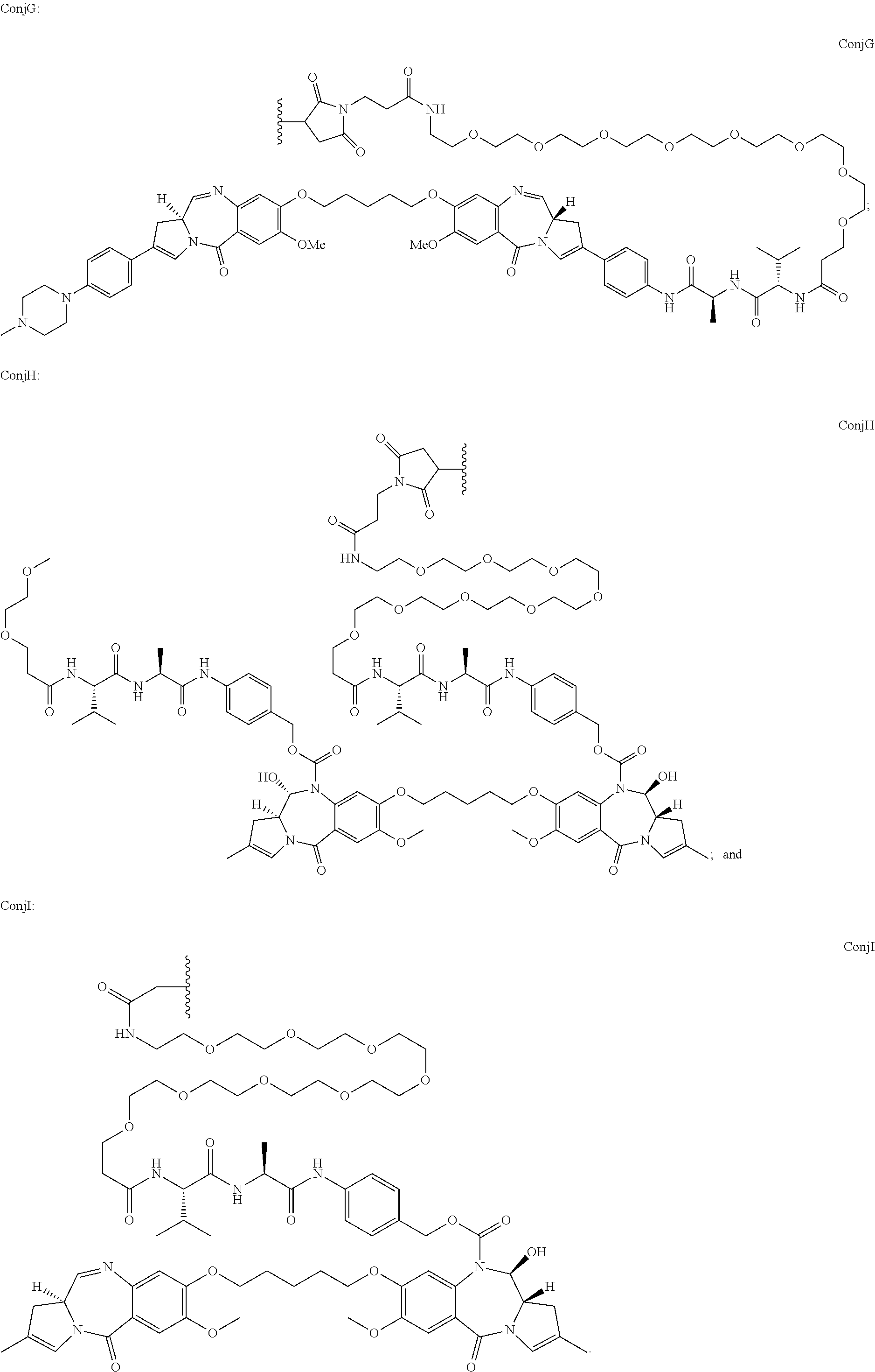

101. The method according to any one of paragraphs 1 to 100 wherein the antibody in an intact antibody.
102. The method according to any one of paragraphs 1 to 101 wherein the antibody is humanised, deimmunised or resurfaced.
103. The method according to any one of paragraphs 1 to 102 wherein the antibody is a fully human monoclonal IgG1 antibody, preferably IgG1,κ.
104. The method according to any one of paragraphs 1 to 100 wherein the antibody is selected from: basiliximab; daclizumab; HuMax-TAC.
105. The method according to any one of paragraphs 101-103, wherein the antibody comprises:
a VH domain comprising a VH CDR1 with the amino acid sequence of SEQ ID NO. 3,
a VH CDR2 with the amino acid sequence of SEQ ID NO. 4, and a VH CDR3 with the amino acid sequence of SEQ ID NO. 5.
   106. The method according to paragraph 105 wherein the antibody comprises a VH domain having the sequence according to SEQ ID NO. 1.
   107. The method according to any one of paragraphs 105 to 106 wherein the antibody comprises:
a VL domain comprising a VL CDR1 with the amino acid sequence of SEQ ID NO. 6,
a VL CDR2 with the amino acid sequence of SEQ ID NO. 7, and a VL CDR3 with the amino acid sequence of SEQ ID NO. 8.
   108. The method according to paragraph 107 wherein the antibody comprises a VL domain having the sequence according to SEQ ID NO. 2.
   109. The method according to any one of paragraphs 1 to 108 wherein the drug loading (p) of drugs (D) to antibody (Ab) is an integer from 1 to about 8.
   110. The method according to paragraph 109, wherein p is 1, 2, 3, or 4.
   111. The method according to paragraph 109 comprising use of a mixture of the antibody-drug conjugate compounds, wherein the average drug loading per antibody in the mixture of antibody-drug conjugate compounds is about 2 to about 5.
   112. The method according to any one of paragraphs 1 to 100 wherein the conjugate is ADCT-301.
   113. A method of selecting a subject for treatment with a conjugate as defined in any one of paragraphs 1 to 112, which method comprises screening said subject to identify the presence of CD25+ve Acute Myeloid Leukemia.
   114 A method of treating a proliferative disease in a subject, said method comprising:
   (i) identifying the presence in the subject of CD25+ve Acute Myeloid Leukemia;
   (ii) administering to the subject an antibody-drug conjugate compound as defined in any one of paragraphs 1 to 112.
   115 The method according to paragraph 113 or paragraph 114 wherein said screening or identifying is performed by means of a companion diagnostic which identifies CD25+ve cells by means of immunohistochemistry.
   116 The method according to any one of paragraphs 1 to 115 wherein said proliferative disease is Hodgkin's lymphoma or non-Hodgkin's lymphoma.
   117 The method of paragraph 116 wherein the non-Hodgkin's lymphoma is selected from: Peripheral T cell lymphoma; Cutaneous T cell lymphoma; Diffuse large B cell lymphoma; Follicular lymphoma; Mantle cell lymphoma; Chronic lymphocytic leukemia; Anaplastic large cell lymphoma; Acute myeloid leukemia; Acute lymphoblastic leukemia such as Philadelphia chromosome-positive ALL (Ph+ALL) or Philadelphia chromosome-negative ALL (Ph−ALL).
   118 The method of paragraph 117 wherein the neoplasm or neoplastic cells are, or are present in, a non-hematological cancer.
   119 The method according to any one of paragraphs 1 to 118 wherein said neoplasm or neoplastic cells are, or are present in, a solid tumor.
   120 The method according to any one of paragraphs 1 to 119 wherein said neoplasm or neoplastic cells are malignant.
   121 The method according to any one of paragraphs 1 to 120 wherein said neoplasm or neoplastic cells are metastatic.
   122. The method according to any one of paragraphs 1 to 121 wherein the active compound is administered to a patient/subject as a single dose of about 32 μg per kilogram body weight of the subject per day.
   123. The method according to any one of paragraphs 1 to 122 wherein at least 50% of subjects/patients achieve CR or CRi 32 following administration of one or more doses of the conjugates.
   124. An antibody-drug conjugate compound as defined in any one of paragraphs 1 to 112 for use in a method of any one of paragraphs 1 to 123.
   125. Use of an antibody-drug conjugate compound as defined in any one of paragraphs 1 to 112 in the preparation of a medicament for use in a method of any one of paragraphs 1 to 123.

### REFERENCES

Adair J R, Howard P W, Hartley J A, Williams D G, and Chester K A (2012). Antibody-drug conjugates—a perfect synergy. Expert Opin Biol Ther 12(9): 1191-1206.
American Cancer Society, (2015). Cancer facts & figures 2015. Accessed on 20 May 2015 at http://www.cancer.org/research/cancerfactsstatistics/cancerfactsfigures2015/.
Breems D A, Van Putten W L, Huijgens P C, Ossenkoppele G J, Verhoef G E, Verdonck L F, et al (2005). Prognostic index for adult patients with acute myeloid leukemia in first relapse. J Clin Oncol 23:1969-1978.
Buckner, J. H. (2010). Mechanisms of impaired regulation by CD4(+)CD25(+)FOXP3(+) regulatory T cells in human autoimmune diseases. Nat Rev Immunol 10(12): 849-859.
Burchill, M. A., J. Yang, K. B. Vang and M. A. Farrar (2007). Interleukin-2 receptor signaling in regulatory T cell development and homeostasis. Immunol Lett 114(1): 1-8.
Cerny, J., H. Yu, M. Ramanathan, G. D. Raffel, W. V. Walsh, N. Fortier, L. Shanahan, E. O'Rourke, J. Bednarik, B. Barton, A. Kroll-Desrosiers, S. Hao, B. Woda, L. Hutchinson, A. M. Evens, A. G. Rosmarin and R. Nath (2013). Expression of CD25 independently predicts early treatment failure of acute myeloid leukaemia (AML). Br J Haematol 160(2): 262-266.
Cockcroft D W, Gault M H (1976). Prediction of creatinine clearance from serum creatinine. Nephron 16(1):31-41.
Ding L, Ley T J, Larson D E, et al (2012). Clonal evolution in relapsed acute myeloid leukaemia revealed by whole-genome sequencing. Nature 481(7382):506-510.
Döhner H, Estey E H, Amadori S, Appelbaum F R, Büchner T, Burnett A K, et al (2015). Diagnosis and management of acute myeloid leukemia in adults: recommendations from an international expert panel, on behalf of the European Leukemia Net. Blood 115(3):453-474.
Gönen M, Sun Z, Figueroa M E, Patel J P, Abdel-Wahab O, and Racevskis J, (2012). CD25 expression status improves prognostic risk classification in AML independent of established biomarkers: ECOG phase 3 trial, E1900. Blood 120(11):2297-2306.
Grimwade D, Walker H, Harrison G, Oliver F, Chatters S, Harrison C J, et al for the Medical Research Council Adult Leukemia Working Party (2001). The predictive value of hierarchical cytogenetic classification in older adults with acute myeloid leukemia (AML): Analysis of 1065 patients entered into the United Kingdom Medical Research Council AML11 trial. Blood 98:1312-1320.
Hartley J A (2011[1]). The development of pyrrolobenzodiazepines as antitumour agents. Expert Opin Investig Drugs 20(6): 733-744.
Hartley J M, Spanswick V J, Hartley J A (2011[2]). Measurement of DNA damage in individual cells using the single cell gel electrophoresis (comet) assay. Methods Mol Biol 731:309-20. Ishikawa F, Yoshida S, Saito Y, Hijikata A, Kitamura H, Tanaka S, et al (2007).
Chemotherapy-resistant human AML stem cells home to and engraft within the bone-marrow endosteal region. Nat Biotechnol 25:1315-1321.
Miltiades P, Lamprianidou E, Vassilakopoulos T P, Papageorgiou S G, Galanopoulos A G, and Vakalopoulou, for the Hellenic MDS Study Group (2014). Expression of CD25 anigen on CD34+ cells is an independent predictor of outcome in late-stage MDS patients treated with azacitidine. Blood Cancer Journal, 4, e187; doi:10.1038/bcj.2014.9 (Letter to the Editor).
National Cancer Institute (2015). SEER Stat Fact Sheets: Acute Myeloid Leukemia (AML), 2015. Accessed on 1 Jun. 2015, at http://seer.cancer.gov/statfacts/html/amyl.html.
Novartis, New Jersey, USA. Prescribing information for Simulect, 2005, accessed on 25 May 2015 at https://www.pharm.us.novartis.com/product/pi/pdf/simulect.pdf.
Oken M M, Creech R H, Tormey D C, et al (1982). Toxicity and response criteria of the Eastern Cooperative Oncology Group. Am J Clin Oncol 5(6):649-55.
Roche Pharmaceuticals, New Jersey, USA. Prescribing Information for Zenapax, 2005, accessed on 25 May 2015 at http://www.gene.com/download/pdf/zenxapax_prescribing.pdf.
Saito Y, Kitamura H, Hijikata A, Tomizawa-Murasawal M, Tanaka S, Takagi S et al, (2010). Identification of therapeutic targets for quiescent, chemotherapy-resistant human leukemia stem cells. Sci Transl Med February 3; 2(17): 17ra9. doi:10.1126/scitranslmed.3000349.
Smith T J (Chair), Khatcheressian J, Lyman G H, Ozer H, Armitage J O, Balducci L, et al (2007). 2006 Update of Recommendations for the Use of White Blood Cell Growth Factors: An Evidence-Based Clinical Practice Guideline. J Clin Onc 24(19):3187-3286.
Vardiman J, Harris N L, Brunning R D (2012): The World Health Organization (WHO) classification of the myeloid neoplasms. Blood 100:2292-2302.
Wu J, Clingen P H, Spanswick V J, Melinas-Gomez M, Meyer T, Puzanov I, et al (2012). Clinical Cancer Research, published online Dec. 18, 2012; DOI: 10.1158/1078-0432.CCR-12-2529.

SEQUENCES
SEQ ID NO. 1 (AB12 VH):
QVQLVQSGAEVKKPGSSVKVSCKASGGTFSRYIINWVRQAPGQG
LEWMGRIIPILGVENYAQKFQGRVTITADKSTSTAYMELSSLRS
EDTAVYYCARKDWFDYWGQGTLVTVSSASTKGPSVFPLA
SEQ ID NO. 2 (AB12 VL):
EIVLTQSPGTLSLSPGERATLSCRASQSVSSYLAWYQQKPGQAP
RLLIYGASSRATGIPDRFSGSGSGTDFTLTISRLEPEDFAVYYC
QQYGSSPLTFGGGTKVEIKRTVAAPSVFIFP
SEQ ID NO. 3 (VH CDR1):
RYIIN
SEQ ID NO. 4 (VH CDR2):
RIIPILGVENYAQKFQG
SEQ ID NO. 5 (VH CDR3):
KDWFDY
SEQ ID NO. 6 (VL CDR1):
RASQSVSSYLA
SEQ ID NO. 7 (VL CDR2):
GASSRAT
SEQ ID NO. 8 (VL CDR3):
QQYGSSPLT

1.-124. (canceled)

125. A method of treating CD25+ve acute myeloid leukemia (AML) in a subject, said method comprising administering to a subject a conjugate of formula L-(D^{L})ₚ wherein D^{L} is of formula I or IL 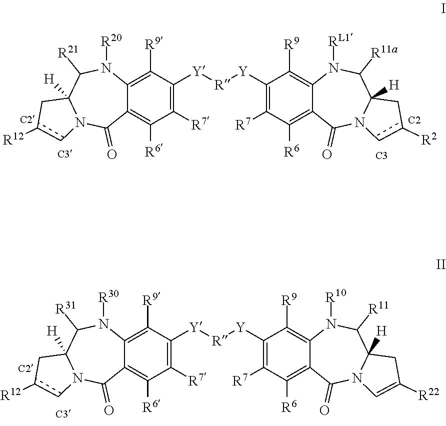
and further wherein:
L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 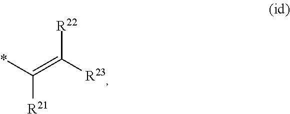
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5; 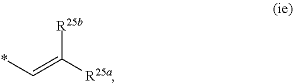
wherein one of R^{25a} and R^{25b} is H and the other is selected from: phenyl, which phenyl is optionally substituted by a group selected from halo, methyl, methoxy; pyridyl; and thiophenyl; and 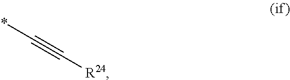
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 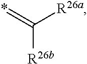
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 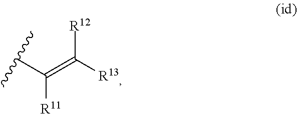
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5; 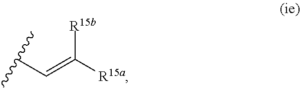
wherein one of R^{15a} and R^{15b} is H and the other is selected from: phenyl, which phenyl is optionally substituted by a group selected from halo, methyl, methoxy; pyridyl; and thiophenyl; and 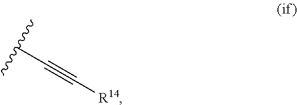
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 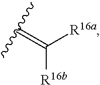
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
R²² is of formula IIIa, formula IIIb or formula IIIc: 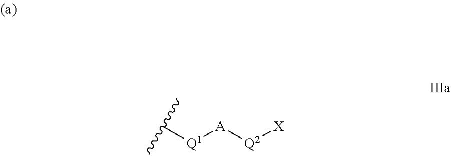
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 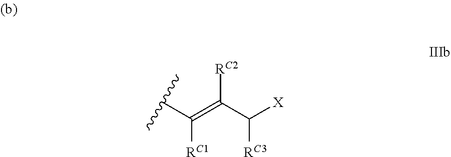
where;
R^{C1}, R^{C2} and R^{C3} are independently selected from H and unsubstituted C₁₋₂ alkyl; 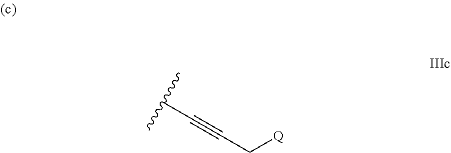
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′} NHNH—R^{L2′}, CONHNH—R^{L2′}, 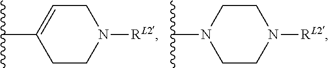
NR^{N}R^{L2′} wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M;
optionally wherein the CD25+ve Acute Myeloid Leukemia comprises both CD25+ve and CD25-ve cells;
and/or the CD25+ve Acute Myeloid Leukemia (AML) is:
(i) refractory AML; or
(ii) relapsed AML.

126. The method of claim 125, wherein:
R⁷ is a C₁₋₄ alkyloxy group, Y is O, R″ is C₃₋₇ alkylene, R⁹ is H, and/or R⁶ is selected from H and halo.

127. The method of claim 125, wherein:
(A) there is a double bond between C2′ and C3′, and R¹² is:
(i) a C₅₋₇ aryl group, which may bear one to three substituent groups selected from methoxy, ethoxy, fluoro, chloro, cyano, bis-oxy-methylene, methyl-piperazinyl, morpholino and methyl-thiophenyl; or
(ii) methyl, ethyl or propyl; or
(iii) cyclopropyl; or
(iv) a group of formula: 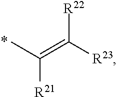
wherein the total number of carbon atoms in the R¹² group is no more than 4; or
(v) the group: 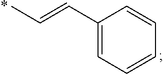
or
(iv) a group of formula: 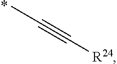
wherein R²⁴ is selected from H and methyl.
OR
(B) there is a single bond between C2′ and C3′, R¹² is R 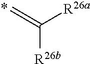
and:
(i) R^{26a} and R^{26b} are both H; or
(ii) R^{26a} and R^{26b} are both methyl; or
(iii) one of R^{26a} and R^{26b} is H, and the other is selected from C₁₋₄ saturated alkyl, C₂₋₃ alkenyl, which alkyl and alkenyl groups are optionally substituted.

128. The method of claim 125, wherein:
(A) there is a double bond between C2 and C3, and R² is:
(i) a C₅₋₇ aryl group which may bear one to three substituent groups selected from methoxy, ethoxy, fluoro, chloro, cyano, bis-oxy-methylene, methyl-piperazinyl, morpholino and methyl-thiophenyl; or
(ii) methyl, ethyl or propyl; or
(iii) cyclopropyl; or
(iv) a group of formula: 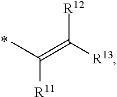
wherein the total number of carbon atoms in the R² group is no more than 4; or
(v) the group: 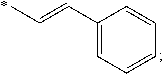
or
(vi) a group of formula: 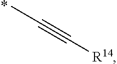
wherein R¹⁴ is selected from H and methyl;
OR
(B) there is a single bond between C2 and C3, R² is 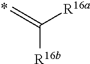
and:
(i) R^{16a} and R^{16b} are both H; or
(ii) R^{16a} and R^{16b} are both methyl; or
(iii) one of R^{16a} and R^{16b} is H, and the other is selected from C₁₋₄ saturated alkyl, C₂₋₃ alkenyl, which alkyl and alkenyl groups are optionally substituted.

129. The method of claim 125, wherein R²⁰ is R^{C}, wherein R^{C} is a group: 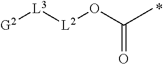
where the asterisk indicates the point of attachment to the N10 position, G² is a terminating group, L³ is a covalent bond or a cleavable linker L¹, L² is a covalent bond or together with OC(═O) forms a self-immolative linker.

130. The method of claim 125, wherein:
(a) R²² is of formula IIIa, and A is phenyl, Q¹ is a single bond, and Q² is a single bond; or
(b) R²² is of formula IIIb, and R^{C1}, R^{C2} and R^{C3} are all H; and
X is NH—R^{L2′}.

131. The method of claim 125, wherein:
(A) R^{6′}, R^{7′}, R^{9′}, and Y′ are the same as R⁶, R⁷, R⁹, and Y;
and/or
(B) L-R^{L1′} or L-R^{L2′} is a group: 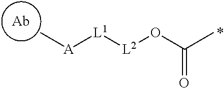
where the asterisk indicates the point of attachment to the PBD, Ab is the antibody, L¹ is a cleavable linker, A is a connecting group connecting L to the antibody, L² is a covalent bond or together with —OC(═O)— forms a self-immolative linker;
optionally wherein,
(i) L¹ comprises a dipeptide and the group —X₁-X₂— in dipeptide, —NH—X₁-X₂—CO—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-,
-Trp-Cit-,
or
(ii) C(═O)O and L² together form the group: 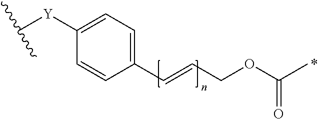
where the asterisk indicates the point of attachment to the PBD, the wavy line indicates the point of attachment to the linker L, Y is NH, O, C(═O)NH or C(═O)O, and n is 0 to 3.

132. The method of claim 125, wherein D^{L} is selected from the group comprising:

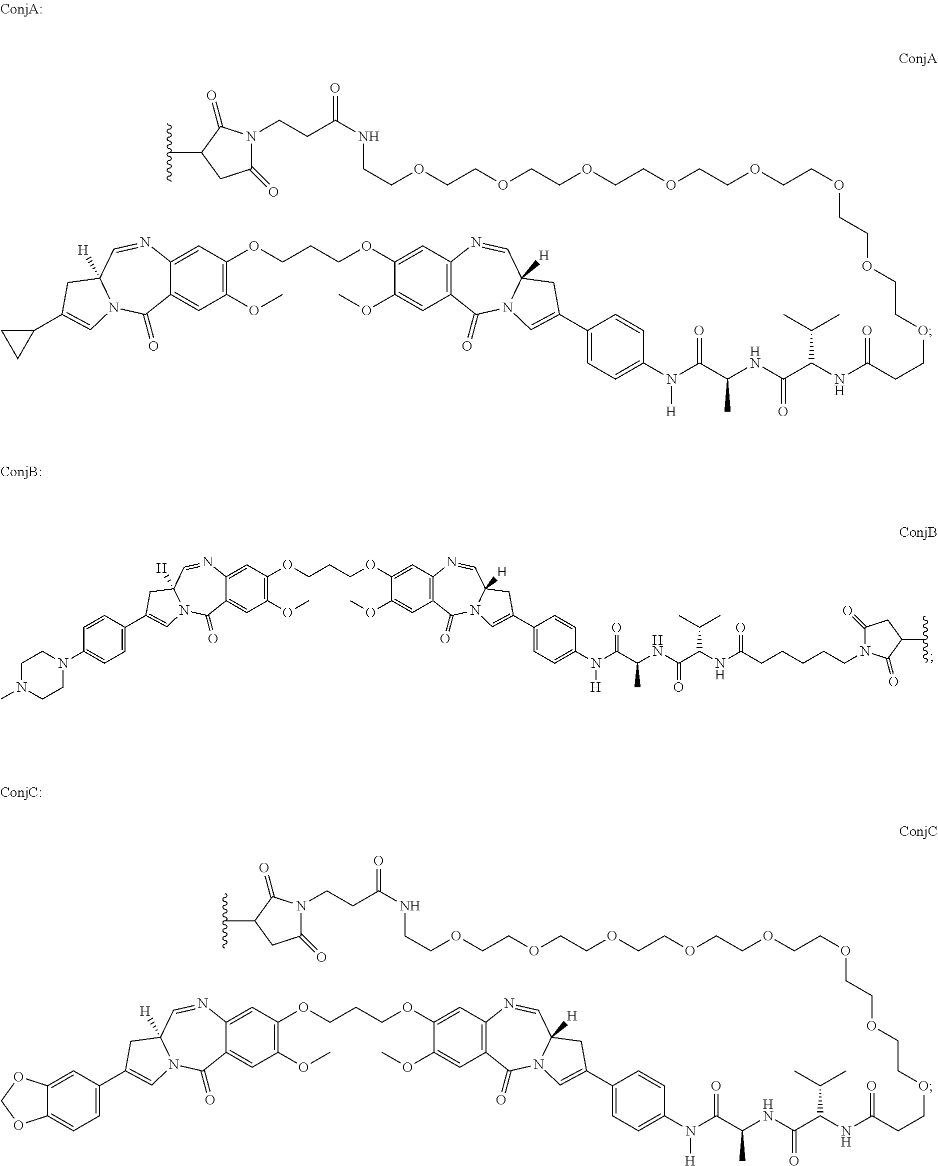

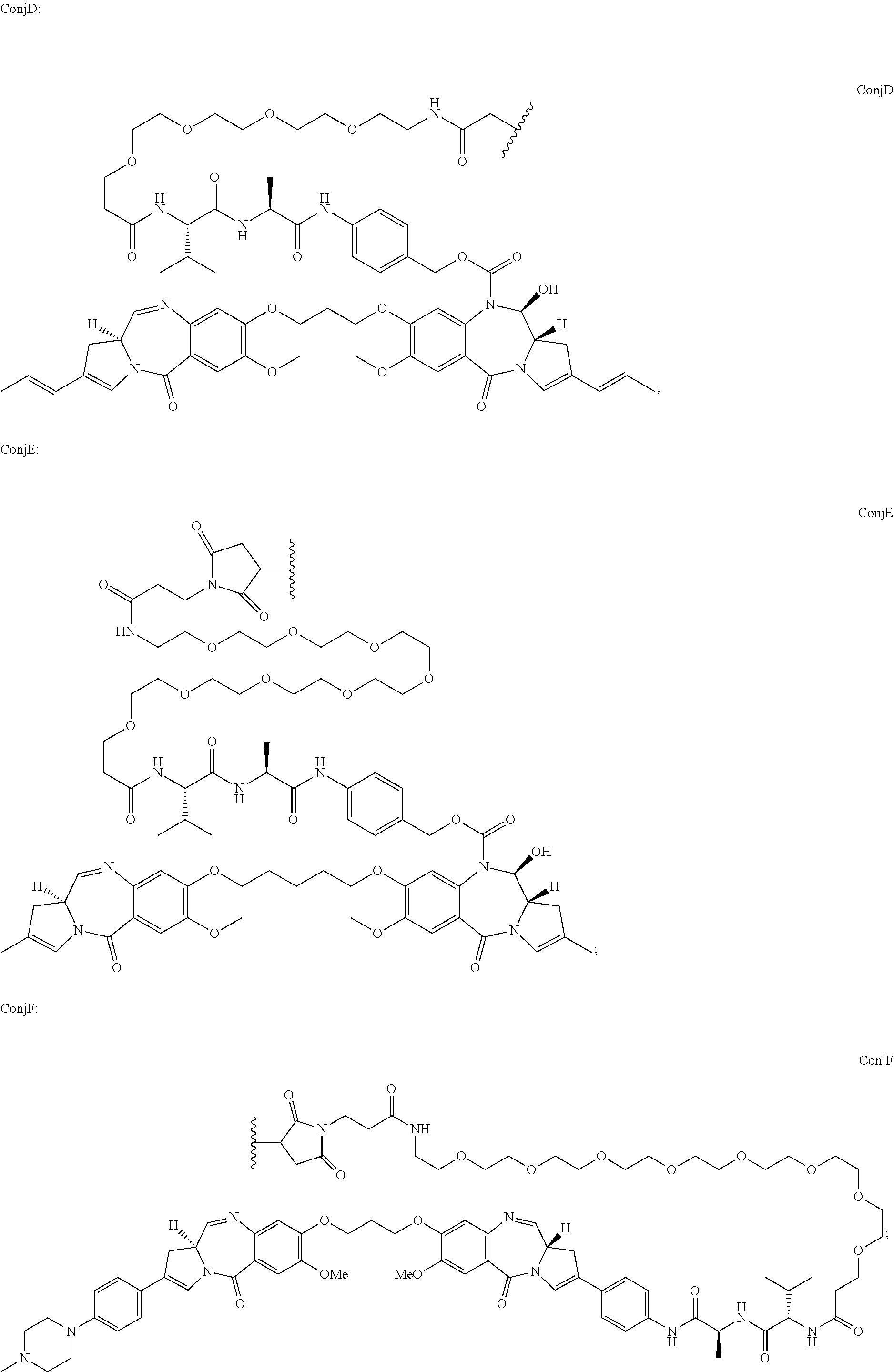

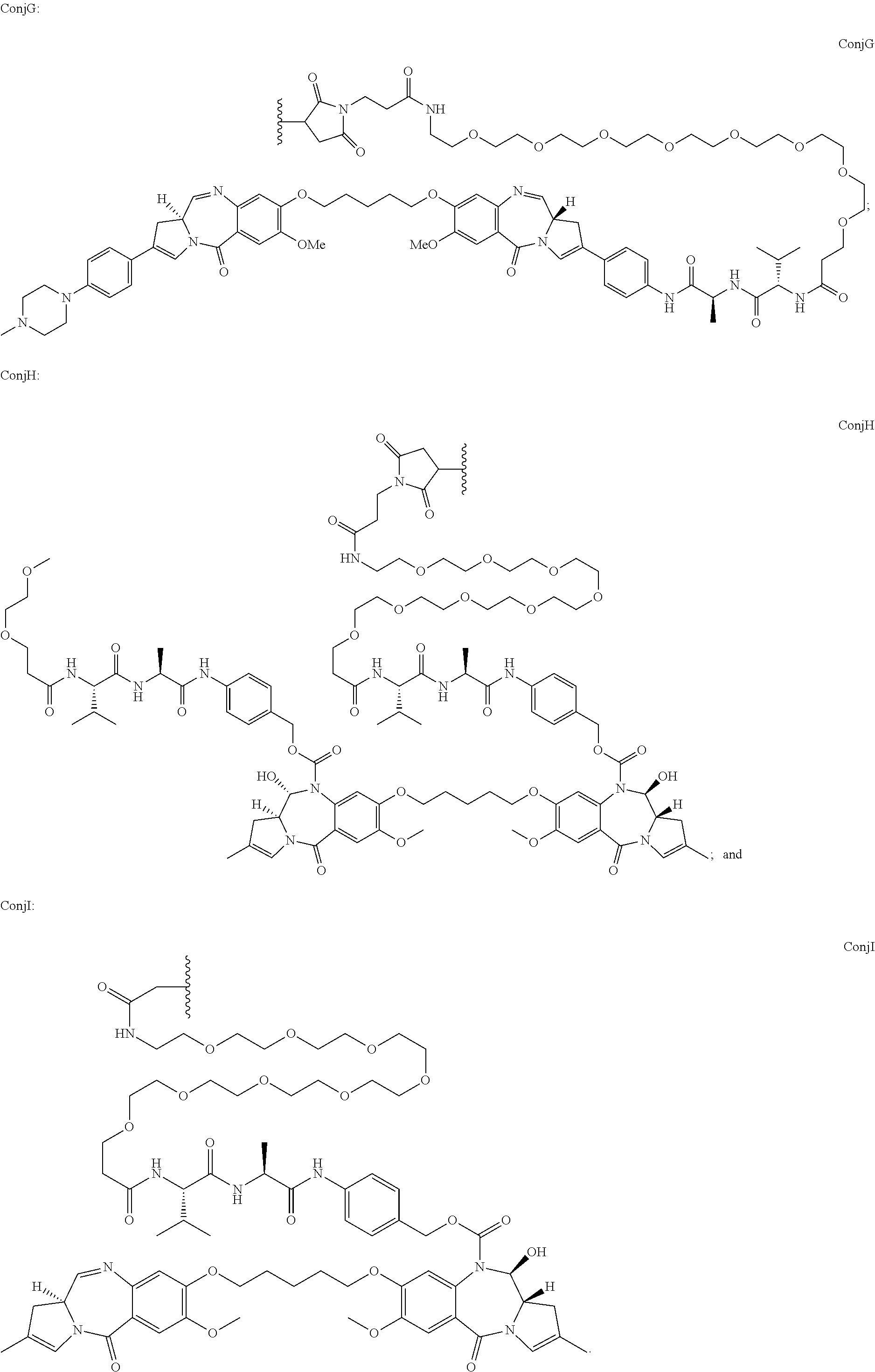

133. The method of claim 125, wherein the antibody comprises:
(A) a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5,
and, optionally, a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8, and/or
(B) a VH domain having the sequence according to SEQ ID NO. 1,
and, optionally further comprises a VL domain having the sequence according to SEQ ID NO. 2.

134. The method of claim 125, wherein:
the drug loading (p) of drugs (D) to antibody (Ab) is an integer from 1 to about 8;
the drug loading (p) of drugs (D) to antibody (Ab) is 1, 2, 3, or 4; or
the average drug loading per antibody in the mixture of antibody-drug conjugate compounds is about 2 to about 5.

135. The method of claim 125, wherein the conjugate is ADCT-301.

136. The method of claim 125, wherein the method further comprises a step of screening a subject to identify the presence of CD25+ve acute myeloid leukemia;
optionally, wherein said screening is performed by means of a companion diagnostic which identifies CD25+ve cells by means of immunohistochemistry.

137. The method of claim 125, wherein said method further comprises:
(i) identifying the presence in the subject of CD25+ve acute myeloid leukemia,
optionally wherein said identifying is performed by means of a companion diagnostic which identifies CD25+ve cells by means of immunohistochemistry; and
(ii) administering to the subject the antibody-drug conjugate compound.

138. The method of claim 125, wherein:
(i) said proliferative disease is Hodgkin's lymphoma or non-Hodgkin's lymphoma, optionally wherein the non-Hodgkin's lymphoma is selected from: Peripheral T cell lymphoma; Cutaneous T cell lymphoma; Diffuse large B cell lymphoma; Follicular lymphoma; Mantle cell lymphoma; Chronic lymphocytic leukemia; Anaplastic large cell lymphoma; Acute myeloid leukemia; Acute lymphoblastic leukemia;
(ii) the neoplasm or neoplastic cells are, or are present in, a non-hematological cancer;
(iii) said neoplasm or neoplastic cells are, or are present in, a solid tumor;
(iv) said neoplasm or neoplastic cells are malignant; or
(v) said neoplasm or neoplastic cells are metastatic.
